(19) United States
(12) Patent Application Publication
(10) Pub. No.: US 2025/0003093 A1
(43) Pub. Date: Jan. 2, 2025

(54) Association-Based Activity Coefficient Model for Electrolyte Solutions

(71) Applicant: Texas Tech University System, Lubbock, TX (US)

(72) Inventors: Chau-Chyun Chen, Lubbock, TX (US); Yu-Jeng Lin, Taipei City (TW)

(21) Appl. No.: 18/293,818

(22) PCT Filed: Aug. 3, 2022
(86) PCT No.: PCT/US2022/039273

§ 371 Date: Jan. 31, 2024

Related U.S. Application Data
(60) Provisional application No. 63/230,291, filed on Aug. 6, 2021.

(51) Int. Cl. C25B 15/02; G16C 20/30

(52) U.S. Cl. CPC C25B 15/02; G16C 20/30

(57) ABSTRACT

A system and method for determining an activity coefficient (γᵢ) for an electrolyte mixture by providing one or more processors, a. memory communicably coupled to the one or more processors and an output device communicably coupled to the one or more processors, calculating, using the one or more processors, the activity coefficient (γᵢ) for the electrolyte mixture based, on association interactions between any species that associate, long-range interactions between ions, and short-range interactions between any species, providing the activity coefficient (γᵢ) for the electrolyte mixture to the output device, and developing a chemical process or a product using the activity coefficient (γᵢ) for the electrolyte mixture.

### CROSS-REFERENCE TO RELATED APPLICATIONS

This application claims priority to U.S. Provisional Application Ser. No. 63/230,291, filed Aug. 6, 2021, the entire contents of which are incorporated herein by reference.

### STATEMENT OF FEDERALLY FUNDED RESEARCH

This invention was made with government support under DE-EE0007888 awarded by the U.S. Department of Energy. The government has certain rights in the invention.

### TECHNICAL FIELD OF THE INVENTION

The present invention relates in general to the field of electrolyte modeling, and more particularly, to association-based activity coefficient model for electrolyte solutions.

### BACKGROUND OF THE INVENTION

Without limiting the scope of the invention, its background is described in connection with modeling electrolyte solutions.

Electrolyte solutions are ubiquitous in the chemical industry and are used in a wide variety of applications. A rigorous and accurate thermodynamic model is the foundation of process modeling, simulation, design, and optimization for electrolyte solutions. Modeling electrolyte solutions is challenging because of the high non-ideality resulted from the charged species. The thermodynamic models need to be able to represent various types of thermodynamic properties and fluid phase equilibria, cover a wide range of concentration and temperature, and extendable to mixed-salt and mixed-solvent systems without excessive parameters.

Substantial advances in modeling electrolyte thermodynamics have been made in the past decades [1]. Pitzer's model and the electrolyte nonrandom two-liquid model (eNRTL) are the two most used models in academia and commercial applications. Pitzer's pioneering model well represents dilute aqueous electrolyte solutions up to 6 molal [2]. However, the model requires high-order parameters for concentrated solutions and complex expressions for mixed-salt systems [3,4]. The eNRTL model improved over Pitzer's model and is applicable to the entire concentration range from infinite dilution to pure salt [5,6]. Employing the local composition theory to account for the complex interactions between ion and water using two binary parameters per salt, eNRTL has been extended to mixed-salt and mixed-solvent systems and successfully applied to model vapor-liquid, liquid-liquid, and solid-liquid phase equilibria [7,8]. However, the modeling results are less satisfactory for the salts that are strongly hydrated such as acids, lithium, calcium, and magnesium salts, especially in concentrated solutions. As shown in FIG. 1, Pitzer's model (red line) overpredicts the activity coefficients of LiCl in the aqueous solution and diverges from the data above 10 m [9]. The extrapolation of eNRTL model with default parameters (blue line) above 6 m underpredicts the activity coefficients. Refitting the eNRTL model (green line) with the data in the entire concentration range up to 19 m still cannot give satisfactory results. The significant deviations suggest that the conventional models cannot adequately represent the water-ion interactions for strongly hydrated ions. The non-ideality due to ion hydration should be explicitly considered and needs additional treatment.

Several activity coefficient models that explicitly consider ion hydration have been proposed [10-15]. Stokes and Robinson first added the hydration correction to the activity coefficient, accounting for the fact that the true water concentration becomes lower as ions are hydrated [10]. Salt-specific hydration numbers were identified by fitting the activity coefficient data. The correction term has been adopted in several activity coefficient models [11,12]. In other proposed models, the hydration process was described as chemical equilibriums between water and ions and was characterized by adjustable chemical equilibrium constants [13-15].

These hydration-based electrolyte models have several limitations even though they have improved the fitting results. First, the hydration correction from Stokes and Robinson has a maximum concentration limit. The assumption that ions are always hydrated with the assigned hydration number regardless of concentration makes the free water molecules depleted as the electrolyte concentration increases [15]. Second, ion-specific hydration parameters could not be identified. Mixing rules were required to scale the hydration contributions from each salt when the model is applied to the mixed-salt systems [16-17]. Finally, the “non-ideality” considered in these models simply comes from the correction of the rational concentration due to hydration. The excess Gibbs free energy resulted from the association interactions remains untouched. A more fundamental model that describes the non-ideality due to hydration is necessary.

The association theory developed by Wertheim is a powerful tool to describe any types of association interactions and is an essential component in the association-based equation-of-state (EOS) models including Statistical Associating Fluid Theory (SAFT) based models [19,20] and Cubic-Plus-Association (CPA) model [21]. The association-based EOS models have been extended to electrolyte solutions by adding the long-range electrostatic interactions between ions. In most of these electrolyte EOS models, the water-ion interactions are accounted for in the short-range physical interactions (i.e., the dispersive interactions in SAFT-based models and the SRK term in CPA model) and neglected in the association interactions [22-25]. The physical picture that the water-water hydrogen bonding network is disrupted by the ion hydration was mostly excluded.

What is needed is a more sophisticated model that describes hydration phenomena.

### SUMMARY OF THE INVENTION

This work presents an association-based activity coefficient model that explicitly considers the solution non-ideality due to associations among ions and solvent species. Built upon the electrolyte Nonrandom Two-liquid model (eNRTL), the model greatly improves the accuracy of eNRTL for strongly associating electrolyte solutions due to presence of ionic species with high surface charge density. The model successfully correlates mean ionic activity coefficients of 46 aqueous single-salt systems from 10 cations and 5 anions at 298.15 K up to their solubility limits. With the ion-specific association parameters identified, the model accurately predicts activity and osmotic coefficients for aqueous mixed-salt systems at 298.15 K. The temperature dependence of the model results has also been examined at 273-373 K. With superior accuracy over a wide range of concentration and temperature, the model represents a major advancement over eNRTL and has a great potential to be a next-generation model for electrolyte solutions.

This work aims to formulate a new activity coefficient model, the association electrolyte model, to better capture the insight and improve the modeling accuracy for strongly hydrated ions in concentrated solutions using the association theory. The excess Gibbs free energy due to ion hydration and ion-pair formation is explicitly considered under the association theory framework. The resulting association electrolyte model improves from prior hydration-based electrolyte models as it applies to the entire concentration range from infinite dilution to pure salt and can be extended to mixed-salt systems without mixing rules. The ion-specific association parameters are identified by fitting the activity coefficient data of aqueous single-salt systems at 298.15 K. The predictive capability of the association electrolyte model is demonstrated by modeling mixed-salt systems using the parameters obtained from single-salt systems. The temperature dependence is examined by correlating the activity coefficient data at 273-373 K. This work shows that the association electrolyte model has a great potential to be a next-generation electrolyte model with superior accuracy and predictive capability over a wide range of concentration and temperature.

In one embodiment, an apparatus, system or computer includes one or more processors, a memory or data storage, and one or more communication interfaces or input/output interfaces, which can be communicably coupled to one or more output device(s) via a network or communications link. The apparatus, system or computer can be used to determine an activity coefficient (γᵢ) for an electrolyte mixture. The one or more processors calculate the activity coefficient (γᵢ) for the electrolyte mixture based on association interactions between any species that associate, long-range interactions between ions, and short-range interactions between any species. The activity coefficient (γᵢ) for the electrolyte mixture is provided to the output device 1608, and a chemical process or a product is developed using the activity coefficient (γᵢ) for the electrolyte mixture.

In one aspect, the electrolyte mixture comprises a single electrolyte solution, an aqueous mixed-salt solution, or a single salt solution. In another aspect, the electrolyte mixture is selected from Table 1, Table 2 or Table 3. In another aspect, the activity coefficient (γᵢ) for the electrolyte mixture is applicable to an entire concentration range from an infinite dilution to a pure salt. In another aspect, the activity coefficient (γᵢ) for the electrolyte mixture is accurate over a temperature range of 273 to 373 K. In another aspect, there are no mixing rules required with any ion-specific association parameters. In another aspect, the activity coefficient (γᵢ) for the electrolyte mixture is calculated using an association electrolyte model comprising.

$$ln {γ}_{i} = ln {γ}_{i}^{ASC} + ln {γ}_{i}^{PDH} + ln {γ}_{i}^{LC}$$

where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory. In another aspect, the association interactions between any species that associate (γᵢ^{ASC}) is calculated using:

$$Ln {γ}_{i}^{A S C} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ]$$

where: superscripts a and d represent electron acceptor site and electron donor site, respectively, Nᵢ is the number of association sites, X_{i,mx} and X_{i,pr} are the unbonded site fractions in the electrolyte mixture and the pure component i, respectively, ρ_{i,mx} and ρ_{i,pr} are the dimensionless molar densities of association sites in the electrolyte mixture and the pure component i, respectively, and rᵢ is the normalized Bondi's volume parameters. In another aspect, wherein rᵢ is specified as 0.76 for water and is assumed to be constants at 0.76 for all the ions. In another aspect, wherein the unbonded site fractions in the electrolyte mixture (X_{i,mx}^{a} and X_{i,mx}^{d}) and the pure component (X_{i,pr}^{a} and X_{i,pr}^{d}) are calculated as:

$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$

$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$

$${X}_{i , pr}^{a} = \frac{1}{1 + {ρ}_{i , pr}^{d} {X}_{i , pr}^{d} {Δ}^{{a}_{i} {d}_{i}}} , and$$

$${X}_{i , pr}^{d} = \frac{1}{1 + {ρ}_{i , pr}^{a} {X}_{i , pr}^{a} {Δ}^{{a}_{i} {d}_{i}}} .$$

In another aspect, wherein the dimensionless molar densities of association sites in the electrolyte mixture (ρ_{i,mx}^{a} and ρ_{i,mx}^{d}) are calculated from the densities in the pure component (ρ_{i,pr}^{a} and ρ_{i,pr}^{d}) and a mole fraction of species i (xᵢ) as:

$${ρ}_{i , mx}^{a} = \frac{{N}_{i}^{a} {x}_{i}}{{\sum }_{j} {r}_{j} {x}_{j}} ,$$

$${ρ}_{i , mx}^{d} = \frac{{N}_{i}^{d} {x}_{i}}{{\sum }_{j} {r}_{j} {x}_{j}} ,$$

$${ρ}_{i , pr}^{a} = \frac{{N}_{i}^{a}}{{r}_{i}} , and$$

$${ρ}_{i , pr}^{d} = \frac{{N}_{i}^{d}}{{r}_{i}} .$$

In another aspect, wherein chemical process or product comprises batteries, crystallization, desalination, distillation, gas refining, ion exchange, petroleum refining, or water processing.

In another embodiment, a computerized method for determining an activity coefficient (γᵢ) for an electrolyte mixture includes providing one or more processors, a memory communicably coupled to the one or more processors and an output device communicably coupled to the one or more processors. The one or more processors calculate the activity coefficient (γᵢ) for the electrolyte mixture based on association interactions between any species that associate, long-range interactions between ions, and short-range interactions between any species. The activity coefficient (γᵢ) for the electrolyte mixture is provided to the output device. A chemical process or a product is developed using the activity coefficient (γᵢ) for the electrolyte mixture. The method can be implemented with a computer program embodied on a non-transitory computer readable storage medium that is executed using one or more processors to perform the method.

In one aspect, the method includes selecting the electrolyte mixture, wherein the electrolyte mixture comprises a single electrolyte solution, an aqueous mixed-salt solution, or a single salt solution. In another aspect, the method includes selecting the electrolyte mixture, wherein the electrolyte mixture is selected from Table 1, Table 2 or Table 3. In another aspect, the activity coefficient (γᵢ) for the electrolyte mixture is applicable to an entire concentration range from an infinite dilution to a pure salt. In another aspect, the activity coefficient (γᵢ) for the electrolyte mixture is accurate over a temperature range of 273 to 373 K. In another aspect, there are no mixing rules required with any ion-specific association parameters. In another aspect, the activity coefficient (γᵢ) for the electrolyte mixture is calculated using an association electrolyte model comprising:

$$ln {γ}_{i} = ln {γ}_{i}^{ASC} + ln {γ}_{i}^{PDH} + ln {γ}_{i}^{LC}$$

where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory. In another aspect, the association interactions between any species that associate (γᵢ^{ASC}) is calculated using:

$$Ln {γ}_{i}^{A S C} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{a} ( \frac{1 - {X}_{j , mx}^{d}}{2} ) ]$$

where: superscripts a and d represent electron acceptor site and electron donor site, respectively, Nᵢ is the number of association sites, X_{i,mx} and X_{i,pr} are the unbonded site fractions in the electrolyte mixture and the pure component i, respectively. ρ_{i,mx} and ρ_{i,pr} are the dimensionless molar densities of association sites in the electrolyte mixture and the pure component i, respectively, and rᵢ is the normalized Bondi's volume parameters. In another aspect, wherein rᵢ is specified as 0.76 for water and is assumed to be constants at 0.76 for all the ions. In another aspect, wherein the unbonded site fractions in the electrolyte mixture (X_{i,mx}^{a}, and X_{i,mx}^{d}) and the pure component (X_{i,pr}^{a} and X_{i,pr}^{d}) are calculated as:

$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$

$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$

$${X}_{i , pr}^{a} = \frac{1}{1 + {ρ}_{i , pr}^{d} {X}_{i , pr}^{d} {Δ}^{{a}_{i} {d}_{i}}} , and$$

$${X}_{i , pr}^{d} = \frac{1}{1 + {ρ}_{i , pr}^{a} {X}_{i , pr}^{a} {Δ}^{{a}_{i} {d}_{i}}} .$$

In another aspect, wherein the dimensionless molar densities of association sites in the electrolyte mixture (ρ_{i,mx}^{a} and ρ_{i,mx}^{d}) are calculated from the densities in the pure component (ρ_{i,pr}^{a} and ρ_{i,pr}^{d}) and a mole fraction of species i (xᵢ) as:

$${ρ}_{i , mx}^{a} = \frac{{N}_{i}^{a} {x}_{i}}{{\sum }_{j} {r}_{j} {x}_{j}} ,$$

$${ρ}_{i , mx}^{d} = \frac{{N}_{i}^{d} {x}_{i}}{{\sum }_{j} {r}_{j} {x}_{j}} ,$$

$${ρ}_{i , pr}^{a} = \frac{{N}_{i}^{a}}{{r}_{i}} , and$$

$${ρ}_{i , pr}^{d} = \frac{{N}_{i}^{d}}{{r}_{i}} .$$

In another aspect, wherein chemical process or product comprises batteries, crystallization, desalination, distillation, gas refining, ion exchange, petroleum refining, or water processing.

### BRIEF DESCRIPTION OF THE DRAWINGS

For a more complete understanding of the features and advantages of the present invention, reference is now made to the detailed description of the invention along with the accompanying figures and in which:

FIG. 1 depicts molality-based mean ionic activity coefficients of LiCl in aqueous solution at 298.15 K, wherein O depicts measured data [9], the red line depicts Pitzer's model, the blue line depicts eNRTL with default parameters fitted with data up to 6 m, and the green line depicts eNRTL refitted with data up to 19 m;

FIGS. 2A-2C depicts schematic diagrams of ion-pairing formations: (a) double-solvent-separated ion pair (2SIP), (b) solvent-shared ion pair (SIP), (c) contact ion pair (CIP), wherein red depicts cation, green depicts anion, and blue depicts solvent;

FIG. 3 is a table depicting the root-mean-square deviations of ln γ_{±m} of 46 single-salt aqueous systems at 298.15 K from combinations of 10 cations and 5 anions, wherein the first row of each salt is in blue corresponding to the eNRTL model, and the second row of each salt is in red corresponding to the association electrolyte model;

FIGS. 4A-4B depict molality-based mean ionic activity coefficients of acids (FIG. 4A) and lithium salts (FIG. 4B) in aqueous solution at 298.15 K, wherein O depicts measured data from sources indicated in Table 1, the solid lines depict the association electrolyte model, and the dotted line depicts the eNRTL model;

FIGS. 5A-5B depict molality-based mean ionic activity coefficients of sodium (FIG. 5A) and potassium salts (FIG. 5B) in aqueous solution at 298.15 K, wherein O depicts measured data from sources indicated in Table 1, and the solid lines depict the association electrolyte model;

FIGS. 6A-6B depict molality-based mean ionic activity coefficients of rubidium (FIG. 6A) and cesium (FIG. 6B) in aqueous solution at 298.15 K, wherein O depicts measured data from sources indicated in Table 1, and the solid lines depict the association electrolyte model.

FIGS. 7A-7B depict molality-based mean ionic activity coefficients of magnesium (FIG. 7A) and calcium (FIG. 7B) in aqueous solution at 298.15 K, wherein O depicts measured data from sources indicated in Table 1, the solid lines depict the association electrolyte model, and the dotted line depicts the eNRTL model;

FIGS. 8A-8B depict molality-based mean ionic activity coefficients of strontium (FIG. 8A) and barium (FIG. 8B) in aqueous solution at 298.15 K, wherein O depicts measured data from sources indicated in Table 1, and the solid lines depict the association electrolyte model;

FIGS. 9A-9B depict contributions to mole fraction-based activity coefficients of electrolyte (γ_{±}) (FIG. 9A) and water (γ_{H}_{₂}_{O}) (FIG. 9B) in aqueous LiCl solution with the association electrolyte model at 298.15 K;

FIG. 10 depicts association strengths at 298.15 K identified from this work with the ionic radii, wherein the error bars are the standard deviations from data regression;

FIG. 11 depicts molality-based mean ionic activity coefficients of chloride salts with various cations in aqueous solution at 298.15 K, wherein O depicts measured data from sources indicated in Table 1, the solid lines depict the association electrolyte model, and the association electrolyte model successfully captures the data following the association strength of cations as LiCl>NaCl>KCl>RbCl>CsCl;

FIG. 12 depicts molality-based mean ionic activity coefficients of HCl in mixed-salt aqueous systems at 298.15 K, wherein each group has a constant total molality of indicated value, O depicts measured data from sources indicated in Table 2, solid lines depict predictions by association electrolyte model, red lines depict HCl—LiCl aqueous system, and blue lines depict HCl—NaCl aqueous system;

FIGS. 13A-13B depict molality-based trace mean ionic activity coefficients of HCl (FIG. 13A) and HBr (FIG. 13B) in mixed-salt aqueous systems at 298.15 K, wherein each mixture contains 0.01 m of HCl or HBr, O depicts measured data from sources indicated in Table 2, and solid lines depict predictions by association electrolyte model;

FIGS. 14A-14B depict molality-based mean ionic activity coefficients of NaCl (FIG. 14A) and LiCl (FIG. 14B) in aqueous solution at 273-373 K, wherein O depicts measured data from sources indicated in Tables 1 and 3, and solid lines depict the association electrolyte model;

FIGS. 15A-15B depict molality-based mean ionic activity coefficients of MgCh (FIG. 15A) and CaCl (FIG. 15B) in aqueous solution at 273-373 K, wherein O depicts measured data from sources indicated in Tables 1 and 3, and solid lines depict the association electrolyte model;

FIG. 16 is a block diagram of an apparatus, system or computer suitable for performing the methods described herein; and

FIG. 17 is a flow chart depicting a computerized method for determining an activity coefficient (γᵢ) for an electrolyte mixture.

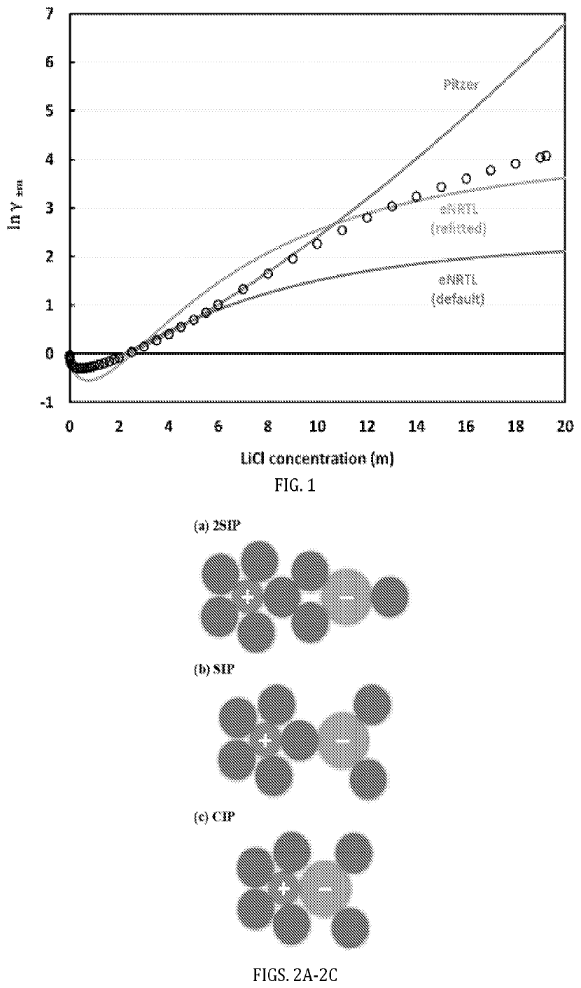

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

This work presents an association-based activity coefficient model that explicitly considers the solution non-ideality due to associations among ions and solvent species. Built upon the electrolyte Nonrandom Two-liquid model (eNRTL), the model greatly improves the accuracy of eNRTL for strongly associating electrolyte solutions due to presence of ionic species with high surface charge density. The model successfully correlates mean ionic activity coefficients of 46 aqueous single-salt systems from 10 cations and 5 anions at 298.15 K up to their solubility limits. With the ion-specific association parameters identified, the model accurately predicts activity and osmotic coefficients for aqueous mixed-salt systems at 298.15 K. The temperature dependence of the model results has also been examined at 273-373 K. With superior accuracy over a wide range of concentration and temperature, the model represents a major advancement over eNRTL and has a great potential to be a next-generation model for electrolyte solutions.

This work aims to formulate a new activity coefficient model, the association electrolyte model, to better capture the insight and improve the modeling accuracy for strongly hydrated ions in concentrated solutions using the association theory. The excess Gibbs free energy due to ion hydration and ion-pair formation is explicitly considered under the association theory framework. The resulting association electrolyte model improves from prior hydration-based electrolyte models as it applies to the entire concentration range from infinite dilution to pure salt and can be extended to mixed-salt systems without mixing rules. The ion-specific association parameters are identified by fitting the activity coefficient data of aqueous single-salt systems at 298.15 K. The predictive capability of the association electrolyte model is demonstrated by modeling mixed-salt systems using the parameters obtained from single-salt systems. The temperature dependence is examined by correlating the activity coefficient data at 273-373 K. This work shows that the association electrolyte model has a great potential to be a next-generation electrolyte model with superior accuracy and predictive capability over a wide range of concentration and temperature.

### Association Electrolyte Model

The association electrolyte model has three contributions to activity coefficient (γᵢ), including the association interactions between any species that associate (γᵢ^{ASC}), the long-range interactions between ions calculated with the Pitzer-Debye-Hückel equation (γᵢ^{PDH}), and the short-range interactions between any species derived from local composition theory (γᵢ^{LC}).

$$ln {γ}_{i} = ln {γ}_{i}^{ASC} + ln {γ}_{i}^{PDH} + ln {γ}_{i}^{LC} \qquad ( 1 )$$

The electrolytes with cation C and anion A are assumed fully dissociated in the solution:

$${C}_{{v}_{c}} {A}_{{v}_{a}} \to {v}_{c} {C}^{{z}_{c} +} + {v}_{a} {A}^{{z}_{a} -} \qquad ( 2 )$$

where: v is the stoichiometric coefficient and z is the charge number; and
the subscripts c and a represent cation and anion, respectively.

The long-range and the short-range interactions are inherited from the eNRTL model. The formulations of γᵢ^{PDH} and γᵢ^{LC} and the model development can be referred to Song and Chen [26].

γᵢ^{PDH} is calculated as the partial molar derivative of G^{ex,PDH}, the Pitzer-Debye-Hückel excess Gibbs energy expression.

$$ln {γ}_{i}^{PDH} = \frac{1}{R T} {( \frac{\partial {G}^{ex , PDH} )}{\partial {n}_{i}} )}_{T , P , {n}_{j \neq i}} \qquad ( 3 )$$

where R is the gas constant, T is the system temperature, P is the system pressure, and i and j are the species indices. G^{ex,PDH} is given in Eq. 4.

$$\frac{{G}^{ex , PDH}}{nRT} = - \frac{4 {A}_{ϕ} {I}_{x}}{ρ} ln [ \frac{1 + ρ {i}_{x}^{1 / 2}}{1 + {ρ ( {l}_{x}^{0} )}^{1 / 2}} ] \qquad ( 4 )$$

with

$${I}_{x} = \frac{1}{2} {\sum }_{i} {z}_{i}^{2} {x}_{i} \qquad ( 5 )$$

where n is the total mole number of the solution, A_{ϕ} is the Debye-Hückel coefficient for the osmotic function, Iₓ is the ionic strength in mole fraction, Iₓ⁰ is Iₓ in pure salt state, ρ is the closest approach parameter, zᵢ is the charge number for species i, and xᵢ is the mole fraction of species i.

γᵢ^{LC} is calculated as the partial molar derivative of G^{ex,LC}, the local composition excess Gibbs energy expression derived from the Nonrandom Two-Liquid theory.

$$ln {γ}_{i}^{LC} = \frac{1}{R T} {( \frac{\partial {G}^{ex , LC} )}{\partial {n}_{i}} )}_{T , P , {n}_{j \neq i}} \qquad ( 6 )$$

G^{ex,LC} is given in Eq. 7.

$$\frac{{G}^{ex , LC}}{RT} = {\sum }_{m} {n}_{m} ( \frac{{\sum }_{i} {X}_{i} {G}_{im} {τ}_{im}}{{\sum }_{i} {X}_{i} {G}_{im}} ) + {\sum }_{c} {z}_{c} {n}_{c} ( \frac{{\sum }_{i \neq c} {X}_{i} {G}_{ic} {τ}_{ic}}{{\sum }_{i \neq c} {X}_{i} {G}_{ic}} ) + {\sum }_{a} {z}_{a} {n}_{a} ( \frac{{\sum }_{i \neq a} {X}_{i} {G}_{ia} {τ}_{ia}}{{\sum }_{i \neq a} {X}_{i} {G}_{ia}} ) \qquad ( 7 )$$

with

$${X}_{i} = {C}_{i} {x}_{i} \qquad ( 8 )$$

$${G}_{i j} = exp ( - {a}_{i j} {τ}_{i j} ) \qquad ( 9 )$$

where m, c, a are the species indices for molecules, cations, and anions, respectively. In Eq. 8, Cᵢ equals zᵢ for ionic species and unity for molecular species. τᵢⱼ's in Eq. 9 are the adjustable NRTL binary interaction parameters while αᵢⱼ's are the NRTL nonrandomness factor parameters, typically set to the value of 0.2.

The formulations of γᵢ^{ASC} are described below.

As the association interactions account for the attractive “chemical” interactions, the short-range interactions calculated by the local composition model are used to capture the remaining repulsive physical interactions. In the local composition model, the interaction energy parameter (τᵢⱼ) quantifies the interaction energy between species i and j and is asymmetric (i.e., τᵢⱼ≠τⱼᵢ). The non-randomness factor (α) is fixed at 0.3 for molecular-molecular pairs and 0.2 for any other species pairs.

The reference state is chosen as symmetric for water and unsymmetric with infinite aqueous dilution for electrolytes.

$${γ}_{{H}_{2} O} ( {x}_{{H}_{2} O} \to 1 ) = 1 \qquad ( 10 )$$

$${γ}_{i}^{*} ( {x}_{{H}_{2} O} \to 1 ) = 1 , i = c , a \qquad ( 11 )$$

The activity coefficients of ions in unsymmetric reference state at infinite aqueous dilution (γ_{c}* and γₐ*) can be calculated as:

$$ln {γ}_{i}^{*} = ln {γ}_{i} - ln {γ}_{i}^{∞} , i = c , a \qquad ( 12 )$$

The γᵢ and γᵢ^{∞} are calculated by a given activity coefficient model. γᵢ^{∞} is the activity coefficient of ions at infinite aqueous dilution:

$${γ}_{i}^{∞} = {γ}_{i} ( {x}_{{H}_{2} O} \to 1 ) , i = c , a \qquad ( 13 )$$

The mole fraction-based (γ_{±}) and molality-based (γ_{±m}) mean ionic activity coefficients can be obtained:

$$ln {γ}_{\pm } = \frac{1}{{v}_{c} + {v}_{a}} ( {v}_{c} ln {γ}_{c}^{*} + {v}_{a} ln {γ}_{a}^{*} ) \qquad ( 14 )$$

$$ln {γ}_{\pm m} = ln {γ}_{\pm } - ln [ 1 + \frac{m ( {v}_{c} + {v}_{a} ) {M}_{s}}{1000} ] \qquad ( 15 )$$

where: m is the molality of electrolytes; and
Mₛ is the molecular weight of solvent.

### Association Theory

The activity coefficient expression of association interactions is based on the association theory, which was first developed by Wertheim and later extended to mixture systems by Chapman [18,19]. Fu et al. further derived the expression of activity coefficients from excess Helmholtz free energy [27]. The activity coefficient expression was later modified to avoid partial derivative calculations [28]. The association theory has been successfully applied to NRTL-SAC and NRTL activity coefficient models to describe the non-ideality due to the hydrogen bonding between molecules in non-electrolyte systems and showed a remarkably better representation of phase equilibria for association systems [29,30].

As described herein, the association theory is applied to calculate the non-ideality resulted from the self-association of water and cross-association of water-ion and cation-anion in aqueous electrolyte solutions using a generalized formulation with two association site types including the electron acceptor and the electron donor. The model formulation can be directly applied to mixed-salt and mixed-solvent systems that involve complex multi-component associations.

$$Ln {γ}_{i}^{ASC} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ] \qquad ( 16 )$$

where: superscripts a and d represent electron acceptor site and electron donor site, respectively;
Nᵢ is the number of association sites;
X_{i,mx} and X_{i,pr} are the unbonded site fractions in the mixture and the pure component i, respectively;
ρ_{i,mx} and ρ_{i,pr} are the dimensionless molar densities of association sites in the mixture and the pure component i, respectively;
rᵢ is the normalized Bondi's volume parameters.

The rᵢ is specified as 0.76 for water and is assumed to be constants at 0.76 for all the ions studied in this work because the regression results are insensitive to the rᵢ of ions.

The unbonded site fractions in the mixture (X_{i,mx}^{a} and X_{i,mx}^{d}) and the pure component (X_{i,pr}^{a} and X_{i,pr}^{d}) are calculated as:

$${X}_{i , mx}^{a} = \frac{1}{1 + {\sum }_{j} {ρ}_{j , mx}^{d} {X}_{j , mx}^{d} {Δ}^{{a}_{i} {d}_{j}}} \qquad ( 17 )$$

$${X}_{i , mx}^{d} = \frac{1}{1 + {\sum }_{j} {ρ}_{j , mx}^{a} {X}_{j , mx}^{a} {Δ}^{{a}_{i} {d}_{j}}} \qquad ( 18 )$$

$${X}_{i , pr}^{a} = \frac{1}{1 + {ρ}_{j , pr}^{d} {X}_{j , pr}^{d} {Δ}^{{a}_{i} {d}_{i}}} \qquad ( 19 )$$

$${X}_{i , p r}^{d} = \frac{1}{1 + {ρ}_{j , pr}^{a} {X}_{j , pr}^{a} {Δ}^{{a}_{i} {d}_{i}}} \qquad ( 20 )$$

The dimensionless molar densities of association sites in the mixture (ρ_{i,mx}^{a} and ρ_{i,mx}^{d}) are calculated from the densities in the pure component (ρ_{i,pr}^{a} and ρ_{i,pr}^{d}) and the mole fraction of species i (xᵢ) as:

$${ρ}_{i , mx}^{a} = \frac{{N}_{i}^{a} {x}_{i}}{{\sum }_{j} {r}_{j} {x}_{j}} \qquad ( 21 )$$

$${ρ}_{i , mx}^{d} = \frac{{N}_{i}^{d} {x}_{i}}{{\sum }_{j} {r}_{j} {x}_{j}} \qquad ( 22 )$$

$${ρ}_{i , pr}^{a} = \frac{{N}_{i}^{a}}{{r}_{i}} \qquad ( 23 )$$

$${ρ}_{i , pr}^{d} = \frac{{N}_{i}^{d}}{{r}_{i}} \qquad ( 24 )$$

The Δ^{α}^{ᵢ}^{d}^{ⱼ} is the binary association strength between the electron acceptor of species i and the electron donor of species j and is characterized by species-specific association strengths (δᵢ^{a} and δᵢ^{d}) with the binary association strength of water self-association Δ^{aH}^{₂}^{OdH}^{₂}^{O}) as a reference.

$${Δ}^{{a}_{i} {d}_{j}} = ( \frac{{δ}_{i}^{a}}{{δ}_{{H}_{2} O}^{a}} ) ( \frac{{δ}_{j}^{d}}{{δ}_{{H}_{2} O}^{d}} ) {Δ}^{{a}_{{H}_{2} O} {d}_{{H}_{2} O}} \qquad ( 25 )$$

The Δ^{aH}^{₂}^{OdH}^{₂}^{O} was obtained by fitting the unbonded site fraction data of pure water at 273-424 K [31].

$${Δ}^{{a}_{{H}_{2} O} {d}_{{H}_{2} O}} ( T ) = 24.2 exp [ 1894 ( \frac{1}{T} - \frac{1}{{T}_{ref}} ) ] \qquad ( 26 )$$

where: Tis the system temperature in Kelvin; and
T_{ref} is the reference temperature at 298.15 K.

The species-specific association strengths of water (δ_{H}_{₂}_{O}^{a} and δ_{H}_{₂}_{O}^{d}) are set as unity and are independent of temperature. The ion-specific association strengths (δᵢ^{a} and δᵢ^{d}) are determined by simultaneously fitting the activity coefficient data of electrolytes in aqueous solutions.

The numbers of association sites for water and each ion used in this work are shown in Table 1. Each water molecule has two electron donors and two electron acceptors from its oxygen and hydrogen atoms, respectively. Cations contain only electron acceptors and anions have only electron donors. The hydration number of ions has been measured experimentally as defined as the number of water molecules near an ion that have lost their translational degrees of freedom and move with the ion as one entity [32]. The hydration numbers measured from various methods generally give reasonable agreement [33,34]. The average hydration numbers of several methods reviewed by Bockris and Conway are adopted for most of the ions studied in this work [33]. The hydration number of strontium has not been reported and is assumed to be the same as barium. Proton was found to hydrate with more than six water molecules at dilute concentration [35,36]. A hydration number of seven is employed for proton in this work. In the trigonal planar and tetrahedral oxyanions, it has been suggested that each oxygen can contribute to two hydrogen bonding donors [37]. For sulfate, there are eight water molecules in the inner shell directly associate with the four oxygens [38]. Nitrate has only three, half of six hydrogen bonding sites are occupied because of the steric effect [39]. The number of association sites of sulfate and nitrate anions is specified as eight and three, respectively.

### Ion-Counterion Associations

Ion associations between cations and anions have been experimentally detected in aqueous solutions even at dilute concentration less than 1 molal [40-43]. The ion pairs can be classified as double-solvent-separated ion pair, (2SIP), solvent-shared ion pair (SIP), and contact ion pair (CIP) as shown in FIGS. 2A-2C [44]. The 2SIP and SIP are associated indirectly through two and one solvent layers, respectively while CIP is formed directly between cations and anions. The existence of ion pairs is far from negligible and can affect the solution thermodynamics [45]. The distribution of ion pairs has been modeled by stepwise association equilibriums as each type of ion pairs was treated as a unique species [44,46]. However, the chemical equilibrium approach requires additional fitting parameters and is even more cumbersome in mixed-salt systems.

In this work, the ion associations are described under the association theory framework without additional parameters. All the ion-pairing types including 2SIP, SIP, and CIP are considered as ions associate with water and counterions simultaneously. It should be noted that the ion complexes formed via coordinate covalent bonds are not included in the ion pairs mentioned above [44]. The ion complexes in covalent bonding nature lose the non-directional electrostatic interactions as the electron density of anion is transferred to the free orbital of cation and should be treated as neutral molecular species with partial dissociation chemistry.

### Data Regression

The ion-specific association strengths (δᵢ^{a} and δᵢ^{d}) and the salt-specific interaction energy parameters (τ_{H}_{₂}_{O-salt}, τ_{salt-H}_{₂}_{O}) are determined by simultaneously fitting the mean ionic activity coefficients data of 46 common salts composed of 10 cations and 5 anions in aqueous solutions at 298.15 K. As listed in Table 1, the mean ionic activity coefficient data cover a wide concentration range up to their solubility limits including data at supersaturated concentration for NaCl and KCl up to 13 molal.

Table 1: Summary of mean ionic activity coefficient data in single-salt aqueous systems at 298.15 K and interaction energy parameters (τ_{H}_{₂}_{O-salt}, τ_{salt-H}_{₂}_{O}) in association electrolyte model.

| | | | Max. | No. of | |
|---|---|---|---|---|---|
| | | | concentration | data | Data |
| Electrolyte | τ_{H}_{₂}_{O - Salt} | τ_{Salt - H}_{₂}_{O} | (m) | points | ref. |
| HCl | 6.6 | −1.1 | 16 | 39 | 9 |
| | | | 18.3 | 14 | 51 |
| HBr | 7.0 | −2.0 | 11 | 34 | 9 |
| | | | 18 | 21 | 52 |
| HI | 7.0 | −2.2 | 10 | 33 | 9 |
| HNO₃ | 3.2 | 5.0 | 28 | 51 | 9 |
| LiCl | 6.6 | −2.2 | 19.2 | 43 | 9 |
| LiBr | 8.1 | −3.3 | 20 | 43 | 9 |
| LiI | 3.4 | 3.6 | 3 | 23 | 9 |
| LiNO₃ | 6.4 | −3.1 | 20 | 43 | 9 |
| Li₂SO₄ | 6.8 | −3.1 | 3.2 | 38 | 53 |
| NaCl | 4.5 | −0.1 | 6.1 | 30 | 9 |
| | | | 13 | 67 | 54 |
| NaBr | 4.3 | −0.3 | 9 | 32 | 9 |
| NaI | 6.1 | −2.3 | 12 | 35 | 9 |
| NaNO₃ | 4.9 | −1.8 | 10.8 | 67 | 55 |
| Na₂SO₄ | 7.5 | −3.5 | 4.4 | 43 | 53 |
| KCl | 7.5 | −3.5 | 5 | 28 | 9 |
| | | | 12.6 | 44 | 54 |
| KBr | 7.1 | −3.4 | 5.5 | 28 | 9 |
| KI | 6.4 | −3.1 | 4.5 | 26 | 9 |
| KNO₃ | 7.6 | −3.4 | 3.5 | 24 | 9 |
| K₂SO₄ | 10.4 | −5.0 | 0.7 | 25 | 53 |
| RbCl | 8.3 | −4.1 | 7.8 | 32 | 9 |
| RbBr | 8.1 | −4.0 | 5 | 27 | 9 |
| RbI | 8.2 | −4.0 | 5 | 27 | 9 |
| RbNO₃ | 7.9 | −3.6 | 4.5 | 26 | 9 |
| Rb₂SO₄ | 8.2 | −4.0 | 1.7 | 31 | 53 |
| CsCl | 8.6 | −4.2 | 11 | 34 | 9 |
| CsBr | 8.6 | −4.2 | 5 | 27 | 9 |
| CsI | 8.4 | −4.1 | 3 | 23 | 9 |
| CsNO₃ | 9.0 | −4.2 | 1.5 | 19 | 9 |
| Cs₂SO₄ | 8.2 | −4.1 | 1.6 | 31 | 53 |
| MgCl₂ | 6.2 | 0.9 | 5.9 | 49 | 56 |
| MgBr₂ | 5.8 | 3.3 | 5.6 | 47 | 56 |
| MgI₂ | 6.0 | −0.8 | 5 | 45 | 56 |
| Mg(NO₃)₂ | 6.7 | −3.0 | 5 | 21 | 57 |
| MgSO₄ | 7.3 | −3.0 | 3.6 | 24 | 58 |
| CaCl₂ | 4.8 | 0.9 | 10 | 65 | 56 |
| CaBr₂ | 7.1 | −2.5 | 9.2 | 62 | 56 |
| CaI₂ | 3.8 | 3.5 | 1.9 | 38 | 56 |
| Ca(NO₃)₂ | 4.1 | −1.2 | 8 | 24 | 59 |
| SrCl₂ | 7.1 | −3.3 | 4 | 42 | 56 |
| SrBr₂ | 4.3 | −1.9 | 2.1 | 40 | 56 |
| SrI₂ | 4.8 | −2.5 | 2 | 38 | 56 |
| Sr(NO₃)₂ | 4.6 | −2.1 | 4 | 19 | 57 |
| BaCl₂ | 5.6 | −2.7 | 1.8 | 36 | 56 |
| BaBr₂ | 7.3 | −3.7 | 2.3 | 42 | 56 |
| BaI₂ | 7.4 | −3.9 | 2 | 38 | 56 |
| Ba(NO₃)₂ | 9.6 | −4.5 | 0.4 | 4 | 57 |

The association electrolyte model was formulated in Aspen Plus® Fortran user model and implemented together with the Data Regression System in Aspen Plus® version 10. The data regression applies the maximum likelihood method to minimize the sum-of-square error objective function.

$${min}_{{δ}_{i}^{a} , {δ}_{i}^{d} , {τ}_{{H}_{2} O - salt ,} {τ}_{salt - {H}_{2} O}} Obj = {\sum }_{k} {( \frac{{Y}_{k}^{model} - {Y}_{k}^{e x p}}{{SD}_{k}} )}^{2} \qquad ( 27 )$$

where: Yₖ^{model} and Yₖ^{exp} are the modeled value and the experimental data of measured property Y respectively;
k is the data point number; and
SDₖ is the standard deviation from experimental measurements.

The SDₖ for the mole fraction and the activity coefficient of electrolyte are assumed to be error-free and 5%, respectively. The interaction energy parameters of the eNRTL model are also regressed using the same data sets for comparison.

### Results and Discussions

The association strengths and the interaction energy parameters obtained from the simultaneous regression are shown in Tables 1 and 2, respectively. The root-mean-square deviations (SDₖ) of variable Y can be calculated as:

$${σ}_{Y} = \sqrt{\frac{{\sum }_{k = 1}^{n} ( {Y}_{k}^{model} - {Y}_{k}^{exp} )}{n}} \qquad ( 28 )$$

where: Yₖ^{model} and Yₖ^{exp} are the modeled value and the experimental data of property Y respectively;
k is the data point number; and
n is the total number of data points.

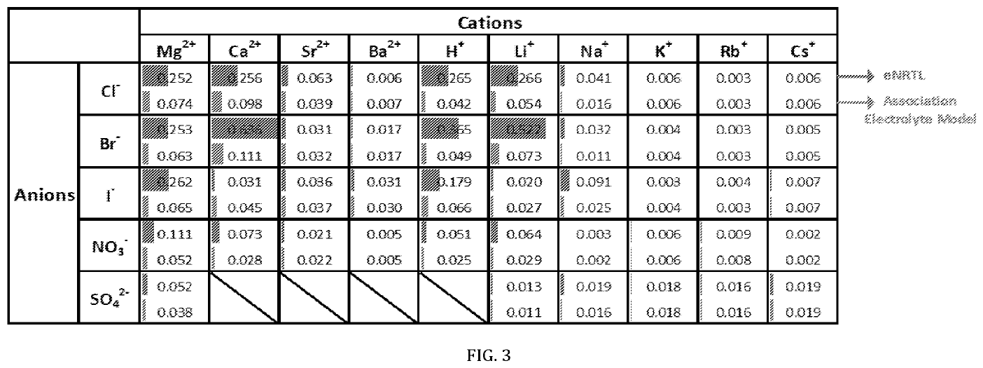

The root-mean-square deviations of logarithm mean ionic activity coefficients (σ_{ln γ±m}) are shown in FIG. 3 and compared between the association electrolyte model and the eNRTL model. With the addition of the ion-specific association strengths, the association electrolyte model provides superior accuracy for all the salts studied in this work. The σ_{ln γ±m} of the association electrolyte model is less than 0.05 in 37 out of 46 salts and less than 0.1 in 45 out of 46 salts. The modeling results are highly accurate considering the ln γ_{±m} can be as high as 6 for the salts that contain ions with high surface charge density such as H⁺, Li⁺, Mg²⁺, and Ca⁺ at ionic strength up to 20 m. The eNRTL model provides a good agreement with data when hydration is not expected to dominate the non-ideality, including the salts with larger ion sizes (e.g., potassium salts and strontium salts) and the salts that only have data at low concentration (e.g., LiI and MgI₂). The association electrolyte model significantly outperforms eNRTL for the strongly hydrated electrolytes such as acids, lithium, magnesium, and calcium salts.

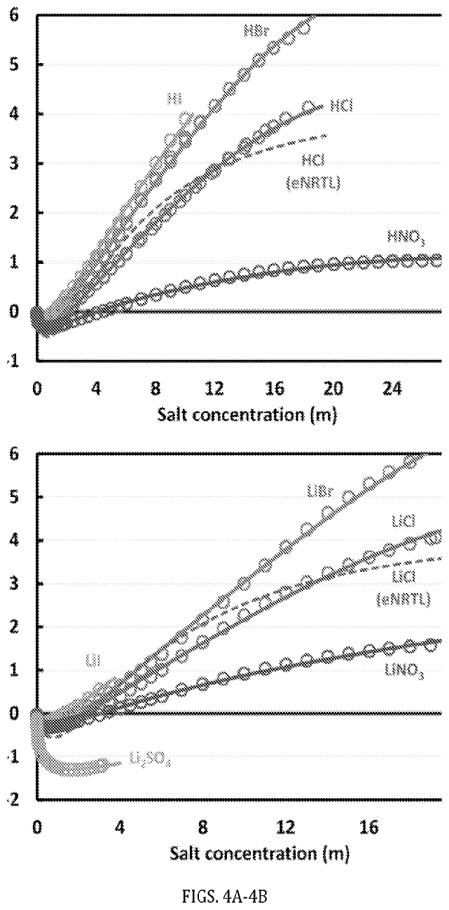

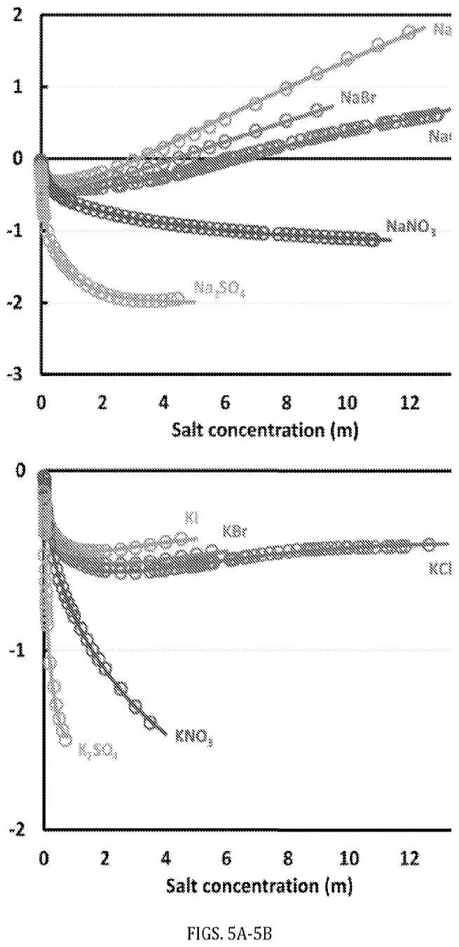

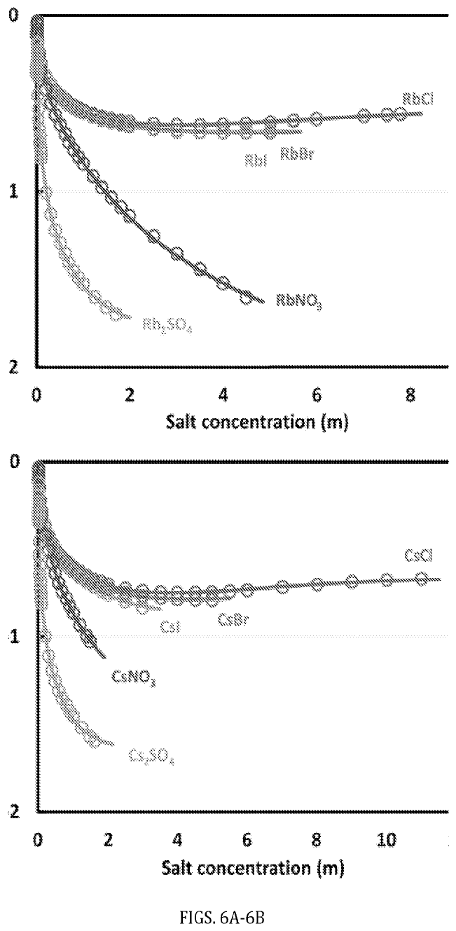

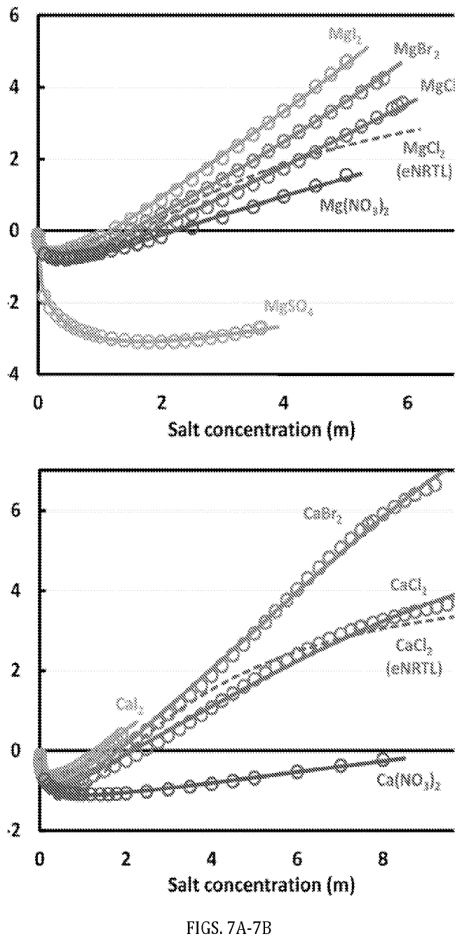

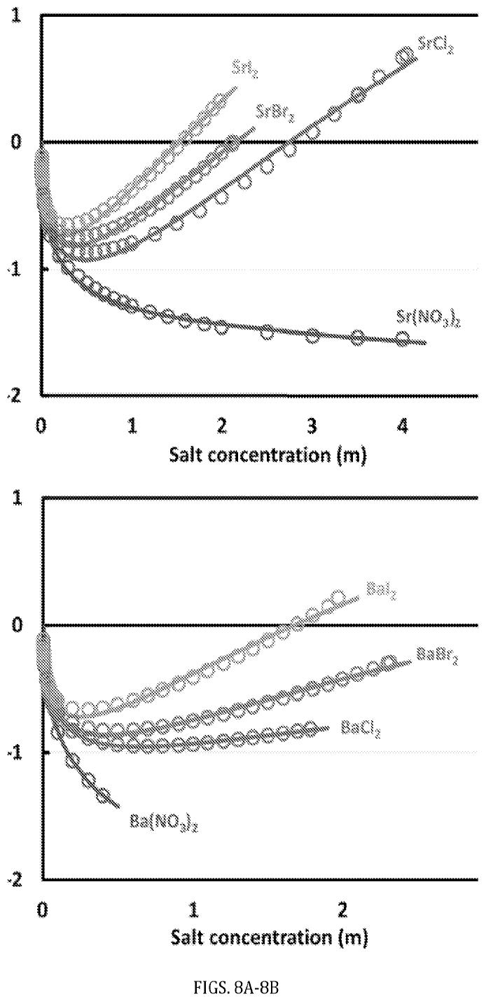

The molality-based mean ionic activity coefficients from the association electrolyte model are compared with measured data in FIGS. 4A-4B, 5A-5B, 6A-6B, 7A-7B and 8A-8B. The activity coefficients of HCl, LiCl, CaCl₂, and MgCl₂ from the eNRTL model are also shown for comparison. The association electrolyte model well correlates the data in the entire concentration range up to their solubility limits while the eNRTL model cannot capture the trend and generally underpredicts the mean ionic activity coefficient at high concentration.

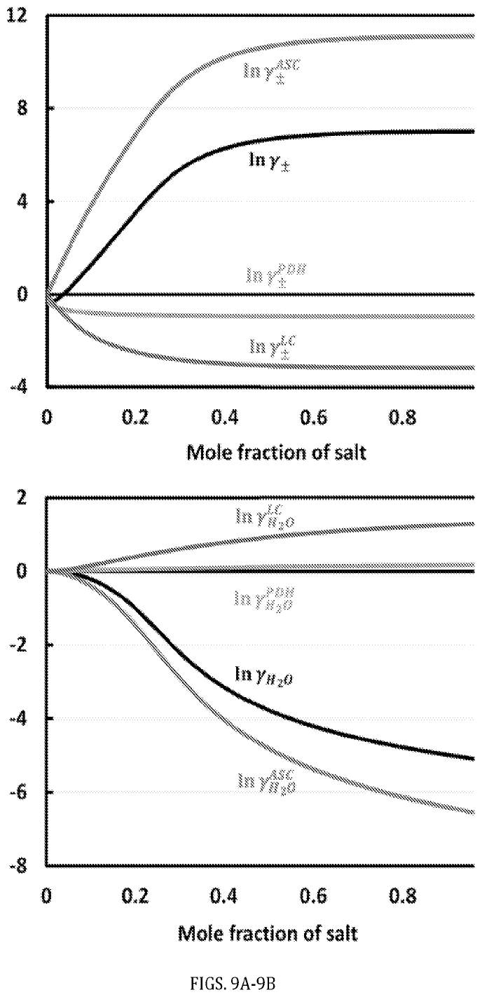

The contributions from γᵢ^{ASC}, γᵢ^{PDH}, and γᵢ^{LC} to the activity coefficients of electrolyte and water are shown in FIGS. 9A-9B using LiCl as an example. The association electrolyte model applies to the entire concentration range from infinite dilution to pure salt without the maximum concentration limit that prior hydration-based electrolyte models have. The contribution of association interactions dominates in the entire concentration range and reaches a plateau at the mole fraction around 0.4 as fewer water molecules are available for hydration.

### Interpretations of Model Parameters

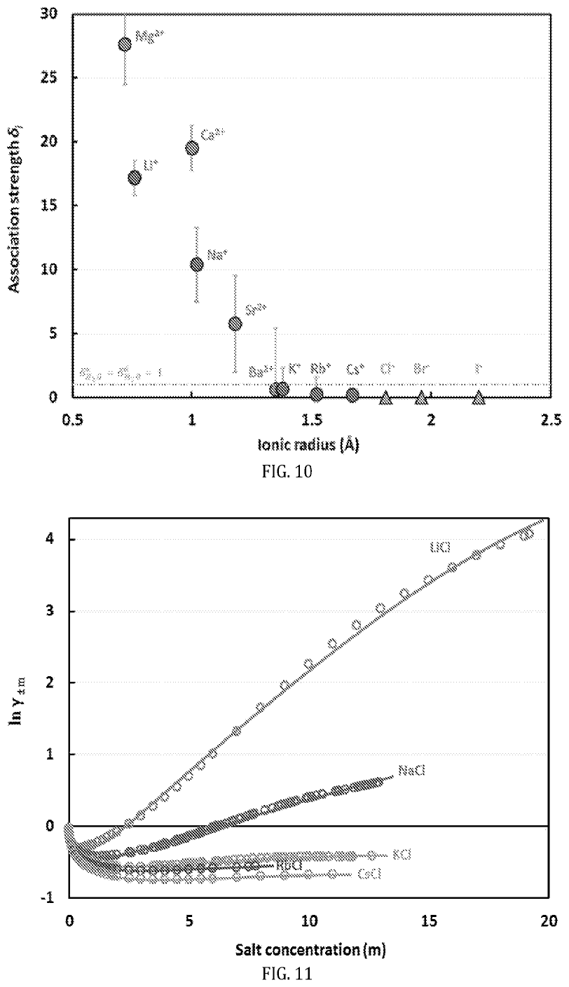

The addition of single association strength for each ion accurately correlates the activity coefficients for all the salts, suggesting the non-ideality resulted from the associations is well captured by the association theory with the ion-specific association parameters. The ions can strongly associate with water and are “structure making” when the association strengths are greater than unity (i.e., the water self-association strength). On the other hand, the ions that have association strengths less than unity are weakly hydrated and are “structure breaking.” The association strengths obtained in this work are qualitatively consistent with prior studies that identified the H⁺, Li⁺, Na⁺, Mg²⁺, and Ca²⁺ to be kosmotropes (structure making) and K⁺, Rb⁺, Cs⁺, Cl⁻, Br⁻, and I⁻ to be chaotropes (structure breaking) using water-water interactions as a critical reference [47,48]. FIG. 10 shows that the association strengths of elemental ions identified in this work are highly correlative with their ionic radii, following the order as Mg²⁺>Ca²⁺>Sr²⁺>Ba²⁺, Li⁺>Na⁺>K⁺>Rb⁺>Cs⁺, and Cl⁻>Br⁻>I⁻.

The association electrolyte model explains the activity coefficient data primarily through the degrees of ion hydration and ion-pair formation. It has been suggested that the rising ionic activity coefficients can be attributed to the extensive hydration of ions and the low/moderate ionic activity coefficients are due to the ion-pair formation [47]. The cations predominantly interact with water molecules when the anions are weaker electron donors than water. In this case, the mean ionic activity coefficients increase with the association strengths of the cations as the cation hydration dominates the non-ideality (i.e., LiX>NaX>KX>RbX>CsX). FIG. 11 shows an example that the association electrolyte model successfully captures this trend when X is Cl. On the other hand, the anions affect the mean ionic activity coefficients mainly through the ion-pair formation because the electron donor of the cations is much stronger than that of water. The anions with greater association strengths are more likely to form more ion pairs and therefore lower the activity coefficients. Consequently, the mean ionic activity coefficients have a reverse order of the association strengths of the anions (i.e., MI>MBr>MCl>MNO₃). This trend is well captured by the association electrolyte model as shown in FIGS. 4A-4B, 5A-5B, 6A-6B, 7A-7B and 8A-8B for all the cations. Finally, when weak anions are paired with weak cations, similar activity coefficients are obtained (i.e., CsCl≈CsBr≈CsI) because neither ion hydration nor ion-pair formation dominates the non-ideality.

### Aqueous Mixed-Salt Systems at 298.15 K

The predictive capability of the association electrolyte model is demonstrated by modeling aqueous mixed-salt systems using the parameters obtained from aqueous single-salt systems. In the prior hydration-based activity coefficient models, ions interacted with water independently without “seeing” each other. Therefore the competition effect between the association species could not be considered and mixing rules had to be applied in mixed-salt systems to scale the hydration contributions from salts by their concentration or ionic strength [14,16,17]. In contrast, the association electrolyte model can be extended to mixed-salt systems without mixing rules as the generalized formulation of the association theory allows all the electron acceptors and donors to interact and compete simultaneously.

Table 2 summarizes the root-mean-square deviations of the mean ionic activity coefficients and the osmotic coefficients (ϕ) predicted by the association electrolyte model for 44 aqueous mixed-salt systems at 298.15 K.

TABLE 2
Predicted activity coefficients and osmotic coefficients
by association electrolyte model for mixed-salt aqueous solutions
at 298.15K; τ_{Salt - Salt} = 0.

| | | No. of | Max. total | | |
|---|---|---|---|---|---|
| | Data | data | ionic | | Data |
| Mixed salts | type | points | strength (m) | σ | ref. |
| HCl + BaCl₂ | ln γ_{±m}^{tr} | 11 | 3 | 0.025 | 49 |
| HCl + CaCl₂ | ln γ_{±m} | 25 | 3 | 0.031 | 60 |
| | ln γ_{±m} | 52 | 5 | 0.068 | 61 |
| HCl + LiCl | ln γ_{±m}^{tr} | 11 | 4 | 0.056 | 49 |
| | ln γ_{±m} | 8 | 3 | 0.051 | 49 |
| | ln γ_{±m} | 18 | 6 | 0.050 | 62 |
| HCl + MgCl₂ | ln γ_{±m} | 25 | 3 | 0.051 | 63 |
| HCl + NaCl | ln γ_{±m}^{tr} | 9 | 3 | 0.020 | 49 |
| | ln γ_{±m} | 11 | 3 | 0.032 | 49 |
| | ln γ_{±m} | 28 | 6 | 0.056 | 62 |
| HCl + SrCl₂ | ln γ_{±m}^{tr} | 12 | 7.5 | 0.030 | 49 |
| HBr + CaBr₂ | ln γ_{±m} | 25 | 2 | 0.031 | 64 |
| HBr + KBr | ln γ_{±m}^{tr} | 11 | 3 | 0.043 | 49 |
| HBr + NaBr | ln γ_{±m}^{tr} | 11 | 3 | 0.031 | 49 |
| HBr + LiBr | ln γ_{±m}^{tr} | 14 | 3 | 0.058 | 49 |
| NaCl + BaCl₂ | ϕ | 48 | 4.8 | 0.048 | 65 |
| NaCl + CaCl₂ | ϕ | 53 | 8.0 | 0.030 | 66 |
| NaCl + CsCl | ϕ | 60 | 6 | 0.118 | 67 |
| NaCl + KCl | ϕ | 85 | 6.6 | 0.073 | 68 |
| NaCl + KNO₃ | ϕ | 18 | 3.7 | 0.043 | 69 |
| NaCl + K₂SO₄ | ϕ | 19 | 3.6 | 0.060 | 70 |
| NaCl + LiCl | ϕ | 36 | 5.8 | 0.016 | 71 |
| | ϕ | 24 | 6 | 0.020 | 72 |
| NaCl + MgCl₂ | ϕ | 15 | 5.9 | 0.021 | 73 |
| | ϕ | 287 | 9.9 | 0.027 | 74 |
| NaCl + Na₂SO₄ | ϕ | 4 | 2.5 | 0.066 | 70 |
| | ϕ | 15 | 9.4 | 0.084 | 73 |
| NaCl + NaNO₃ | ln γ_{±m} | 58 | 6 | 0.067 | 75 |
| | ϕ | 17 | 5.7 | 0.033 | 69 |
| NaNO₃ + KCl | ϕ | 25 | 5.8 | 0.052 | 69 |
| NaNO₃ + KNO₃ | ϕ | 13 | 3.7 | 0.003 | 69 |
| NaNO₃ + LiNO₃ | ϕ | 26 | 8.3 | 0.010 | 71 |
| NaNO₃ + NaBr | ln γ_{±m}^{tr} | 8 | 6 | 0.021 | 75 |
| | ln γ_{±m} | 188 | 6 | 0.021 | 75 |
| Na₂SO₄ + KCl | ϕ | 20 | 4.4 | 0.040 | 70 |
| Na₂SO₄ + K₂SO₄ | ϕ | 18 | 3.6 | 0.041 | 70 |
| Na₂SO₄ + MgSO₄ | ϕ | 15 | 6.1 | 0.051 | 73 |
| KCl + BaCl₂ | ϕ | 52 | 4.7 | 0.028 | 76 |
| KCl + CsCl | ϕ | 50 | 5 | 0.010 | 77 |
| KCl + LiCl | ϕ | 35 | 5 | 0.018 | 72 |
| KCl + KNO₃ | ϕ | 14 | 3.7 | 0.014 | 69 |
| KCl + K₂SO₄ | ϕ | 19 | 2.3 | 0.022 | 70 |
| KCl + SrCl₂ | ϕ | 86 | 7.8 | 0.045 | 78 |
| LiCl + BaCl₂ | ϕ | 23 | 4.3 | 0.018 | 79 |
| LiCl + CsCl | ϕ | 35 | 6 | 0.059 | 77 |
| LiCl + LiNO₃ | ϕ | 27 | 10 | 0.046 | 72 |
| MgCl₂ + CaCl₂ | ϕ | 78 | 12.2 | 0.079 | 80 |
| MgCl₂ + Ca(NO₃)₂ | ϕ | 24 | 20.3 | 0.040 | 81 |
| Mg(NO₃)₂ + CaCl₂ | ϕ | 35 | 16.7 | 0.040 | 81 |
| Mg(NO₃)₂ + Ca(NO₃)₂ | ϕ | 15 | 16.6 | 0.066 | 81 |
| MgCl₂ + Mg(NO₃)₂ | ϕ | 23 | 14.4 | 0.153 | 81 |
| MgCl₂ + MgSO₄ | ϕ | 14 | 3.4 | 0.134 | 73 |
| CaCl₂ + Ca(NO₃)₂ | ϕ | 17 | 21.5 | 0.018 | 81 |
| CsCl + BaCl₂ | ϕ | 27 | 4.1 | 0.018 | 79 |

The interaction energy parameters between the salts (τ_{Salt-Salt}) are specified as zero. The data cover a wide range of ionic strength up to 22 m. The mixtures include various combinations of strongly and weakly hydrated salts with and without common cations/anions. The Finyam and do are less than 0.05 in 35 out of 54 data sets and are less than 0.1 in 51 out of 54 data sets. The association electrolyte model provides excellent predictive capability with root-mean-square deviations comparable to that of aqueous single-salt systems.

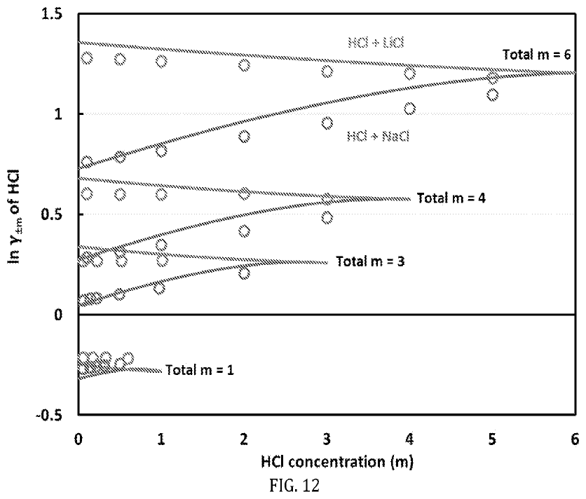

Harned and coworkers have systematically measured the activity coefficients of HCl in the presence of added salts in aqueous solutions at 298.15 K at constant total molality [49]. Harned's rule has emerged from these measurements: the logarithm of the mean ionic activity coefficient of the electrolyte varies linearly with its concentration under the condition of constant total molality. FIG. 12 compares the mean ionic activity coefficients of HCl in the aqueous mixtures with NaCl or LiCl at total molality from 1 to 6 m. The association electrolyte model not only captures the linearity of Harned's rule but also quantitatively predicts the mean ionic activity coefficients of HCl.

Molality-based trace mean ionic activity coefficient (γ_{±m}^{tr}) is a critical thermodynamic property that describes the non-ideality of an electrolyte at trace concentration. The capability of predicting the trace activity coefficients has been emphasized when assessing electrolyte models

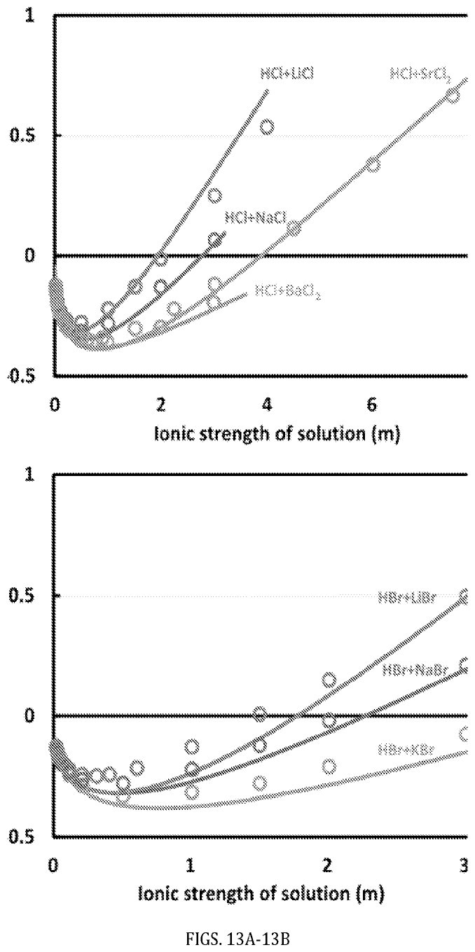

FIGS. 13A-13B compares the measured and the predicted trace activity coefficients of HCl and HBr at trace concentration (0.01 m) with added salts in aqueous solutions at 298.15 K. The association electrolyte model again accurately predicts the trace activity coefficients with σ_{ln γ}_{_{±}}_{^{tr}} at 0.02-0.06.

### Temperature Dependence of Association Strengths

The associations among ions and water molecules are expected to be weakened as temperature increases because the association equilibrium is entropically unfavorable. The mean ionic activity coefficient data generally decrease with increasing temperature, implying the ionic hydration is less significant at elevated temperatures. The γᵢ^{PDH} is intrinsically dependent on temperature and has no adjustable parameters. Additional adjustable parameters are added to the γᵢ^{ASC} and γᵢ^{LC} contributions to account for the temperature dependence. The number of association sites is assumed to be independent of temperature and the hydration numbers at 298.15 K are employed. The temperature dependence of γᵢ^{ASC} is described solely by the association strength using an Arrhenius-type equation with the ion-specific parameters Aᵢ^{a} and Aᵢ^{d}.

$${δ}_{i}^{a} ( T ) = {δ}_{i}^{a} ( {T}_{ref} ) exp [ {A}_{i}^{a} ( \frac{1}{T} - \frac{1}{{T}_{ref}} ) ] \qquad ( 29 )$$

$${δ}_{i}^{d} ( T ) = {δ}_{i}^{d} ( {T}_{ref} ) exp [ {A}_{i}^{d} ( \frac{1}{T} - \frac{1}{{T}_{ref}} ) ] \qquad ( 30 )$$

where δᵢ^{a}(T_{ref}) and δᵢ^{d}(T_{ref}) are the association strengths at reference temperature 298.15 K reported in Table 1. The temperature dependence of the interaction energy parameters follows the expression in the original eNRTL model with one adjustable parameter (Bᵢⱼ) for each τᵢⱼ[6].

$${τ}_{ij} ( T ) = {τ}_{i j} ( {T}_{ref} ) + {B}_{ij} ( \frac{1}{T} - \frac{1}{{T}_{ref}} ) \qquad ( 31 )$$

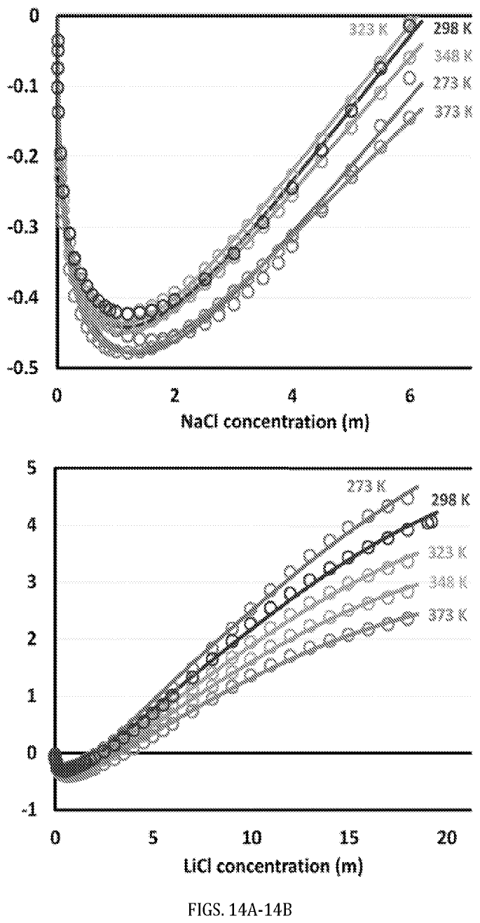

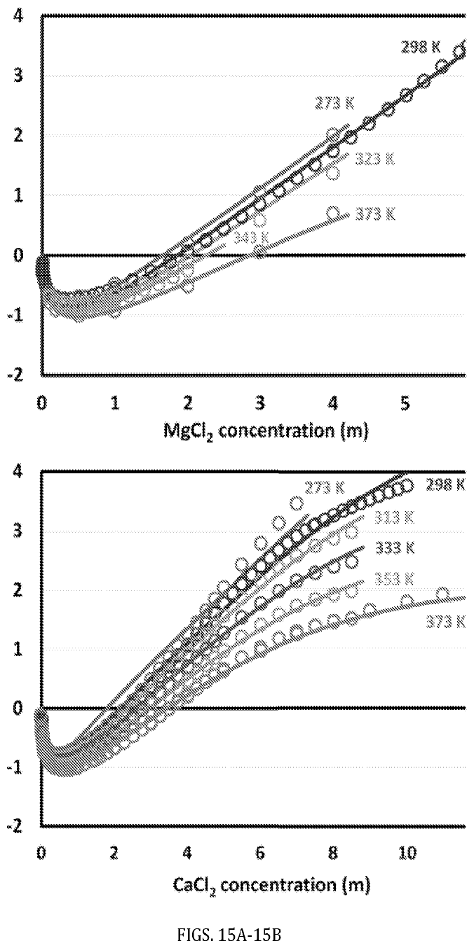

The activity coefficient data of NaCl, HCl, LiCl, MgCl₂, and CaCl₂ aqueous single-salt systems are correlated to demonstrate the temperature dependence. Chloride ion is expected to have little contribution to the temperature dependence because of its weak association strength identified at 298.15 K. The Aᵢ^{d} of chloride is specified as zero. The Aᵢ^{d} for each cation and the B_{H}_{₂}_{O-Salt} and B_{Salt-H}_{₂}_{O} for each salt are determined by fitting the activity coefficient data at 273-373 K with maximum ionic strengths from 6 to 36 m. The results are summarized in Table 3 and plotted in FIGS. 14A-14B and 15A-15B.

TABLE 3
Modeling results of association electrolyte model at
273-373 K and temperature dependence parameters for
association strengths (Aᵢ^{a}, Aᵢ^{d}) and interaction
energy parameters (B_{H}_{₂}_{O}_{-Salt}, B_{Salt-H}_{₂}_{O}).

| | | Max. ionic | | | | | | |
|---|---|---|---|---|---|---|---|---|
| Salt | T (K) | strength (m) | Aᵢ^{a} | Aᵢ^{d} | B_{H}_{₂}_{O-Salt} | B_{Salt-H}_{₂}_{O} | σ_{lnγ±m} | Data ref. |
| HCl | 273-348 | 16 | 1212 | 0 | 3717 | −1855 | 0.030 | 82, 83 |
| LiCl | 273-373 | 18 | 1383 | 0 | 4524 | −2020 | 0.072 | 84 |
| NaCl | 273-373 | 6 | 1592 | 0 | 4964 | −2995 | 0.011 | 85, 86 |
| MgCl₂ | 273-373 | 12 | 2504 | 0 | 3737 | −3759 | 0.072 | 87, 88 |
| CaCl₂ | 273-373 | 36 | 1997 | 0 | 3750 | −3724 | 0.096 | 89, 90 |

The association electrolyte model well correlates the activity coefficient data at 273-373 K and gives 0.011-0.096 of σ_{ln γ}_{_{±m}}, which is comparable to the deviations at 298.15 K. The association electrolyte model provides a consistent accuracy across a wide range of temperature and concentration with a minimal number of temperature dependence parameters required.

### CONCLUSIONS

Explicitly considering the ion hydration and ion-pair formation using the association theory, the association electrolyte model accurately correlates the mean ionic activity coefficients of 46 common aqueous single-salt systems at 298.15 K up to high concentrations at their solubility limits. For strongly associating electrolyte systems, the contribution from the associations dominates the system non-ideality over the contributions from the long-range interactions and the short-range physical interactions. With the addition of the association term and the corresponding association parameters, the association model significantly improves the accuracy over eNRTL for the strongly hydrated acids, lithium, calcium, and magnesium salts. The association strengths identified in this work are qualitatively consistent with prior experimental findings and follow the ionic radii. The association model accurately predicts the activity coefficients and the osmotic coefficients for 44 aqueous mixed-salt systems at 298.15 K using the parameters obtained from aqueous single-salt systems. Finally, with the temperature dependence association strength parameters, the association model accurately correlates the activity coefficients from 273 to 373 K for aqueous single-salt systems. With improved physical insights and superior accuracy and predictive capability over a wide range of concentration and temperature, the association model has a great potential to be a next-generation model for electrolyte solutions. The association model can be extended for mixed-solvent electrolyte systems.

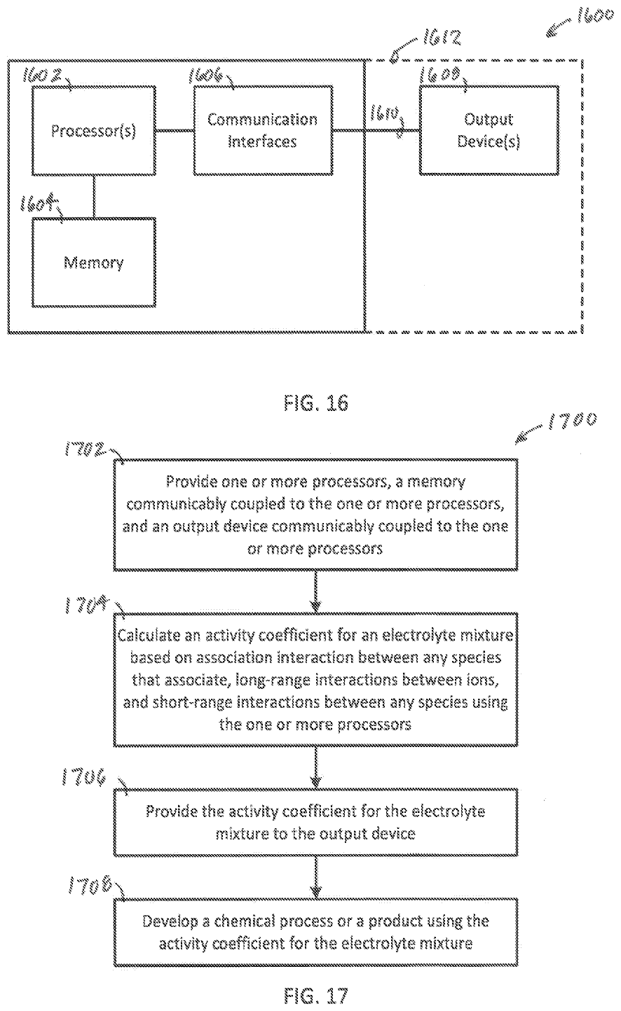

Some other embodiments of the present invention will now be described with respect to FIGS. 16-17. FIG. 16 is a block diagram of an apparatus, system or computer 1600, such as a workstation, laptop, desktop, tablet computer, mainframe, or other single or distributed computing platform suitable for performing the methods described herein. Note that the components can be integrated into a single device or communicably coupled to one another via a network. The apparatus, system or computer 1600 includes one or more processors 1602, a memory or data storage 1604, and one or more communication interfaces or input/output interfaces 1606, which can be communicably coupled to one or more output device(s) 1608 (e.g., printer, internal or external data storage device, display or monitor, remote database, remote computer, etc.) via a network or communications link 1610 (e.g., wired, wireless, optical, etc.). The one or more output device(s) 1608 can be integrated into the computer 1600 as indicated by the dashed line 1612.

The apparatus, system or computer 1600 can be used to determine an activity coefficient (γᵢ) for an electrolyte mixture. The one or more processors calculate the activity coefficient (γᵢ) for the electrolyte mixture based on association interactions between any species that associate, long-range interactions between ions, and short-range interactions between any species. The activity coefficient (γᵢ) for the electrolyte mixture is provided to the output device 1608, and a chemical process or a product is developed using the activity coefficient (γᵢ) for the electrolyte mixture.

In one aspect, the electrolyte mixture comprises a single electrolyte solution, an aqueous mixed-salt solution, or a single salt solution. In another aspect, the electrolyte mixture is selected from Table 1, Table 2 or Table 3. In another aspect, the activity coefficient (γᵢ) for the electrolyte mixture is applicable to an entire concentration range from an infinite dilution to a pure salt. In another aspect, the activity coefficient (γᵢ) for the electrolyte mixture is accurate over a temperature range of 273 to 373 K. In another aspect, there are no mixing rules required with any ion-specific association parameters. In another aspect, the activity coefficient (γᵢ) for the electrolyte mixture is calculated using an association electrolyte model comprising:

$$ln {γ}_{i} = ln {γ}_{i}^{ASC} + ln {γ}_{i}^{PDH} + ln {γ}_{i}^{LC}$$

where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory. In another aspect, the association interactions between any species that associate (γᵢ^{ASC}) is calculated using:

$$Ln {γ}_{i}^{ASC} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ]$$

where: superscripts a and d represent electron acceptor site and electron donor site, respectively, Nᵢ is the number of association sites, X_{i,mx} and X_{i,pr} are the unbonded site fractions in the electrolyte mixture and the pure component i, respectively, ρ_{i,mx} and ρ_{i,pr} are the dimensionless molar densities of association sites in the electrolyte mixture and the pure component i, respectively, and rᵢ is the normalized Bondi's volume parameters. In another aspect, wherein rᵢ is specified as 0.76 for water and is assumed to be constants at 0.76 for all the ions. In another aspect, wherein the unbonded site fractions in the electrolyte mixture (X_{i,mx}^{a} and X_{i,mx}^{d}) and the pure component (X_{i,pr}^{a} and X_{i,pr}^{d}) are calculated as:

$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$

$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$

$${X}_{i , pr}^{a} = \frac{1}{1 + {ρ}_{i , pr}^{d} {X}_{i , pr}^{d} {Δ}^{{a}_{i} {d}_{i}}} , and$$

$${X}_{i , pr}^{d} = \frac{1}{1 + {ρ}_{i , pr}^{a} {X}_{i , pr}^{a} {Δ}^{{a}_{i} {d}_{i}}} .$$

In another aspect, wherein the dimensionless molar densities of association sites in the electrolyte mixture (ρ_{i,mx}^{a} and ρ_{i,mx}^{d}) are calculated from the densities in the pure component (ρ_{i,pr}^{a} and ρ_{i,pr}^{d}) and a mole fraction of species i (xᵢ) as:

$${ρ}_{i , mx}^{a} = \frac{{N}_{i}^{a} {x}_{i}}{{\sum }_{j} {r}_{j} {x}_{j}} ,$$

$${ρ}_{i , mx}^{d} = \frac{{N}_{i}^{d} {x}_{i}}{{\sum }_{j} {r}_{j} {x}_{j}} ,$$

$${ρ}_{i , pr}^{a} = \frac{{N}_{i}^{a}}{{r}_{i}} , and$$

$${ρ}_{i , pr}^{d} = \frac{{N}_{i}^{d}}{{r}_{i}} .$$

In another aspect, wherein chemical process or product comprises batteries, crystallization, desalination, distillation, gas refining, ion exchange, petroleum refining, or water processing.

FIG. 17 is a flow chart depicting a computerized method 1700 for determining an activity coefficient (γᵢ) for an electrolyte mixture. One or more processors, a memory communicably coupled to the one or more processors and an output device communicably coupled to the one or more processors are provided in block 1702. The one or more processors calculate the activity coefficient (γᵢ) for the electrolyte mixture based on association interactions between any species that associate, long-range interactions between ions, and short-range interactions between any species in block 1704. The activity coefficient (γᵢ) for the electrolyte mixture is provided to the output device in block 1706. A chemical process or a product is developed using the activity coefficient (γᵢ) for the electrolyte mixture in block 1708. The method 1700 can be implemented with a computer program embodied on a non-transitory computer readable storage medium that is executed using one or more processors to perform the method 1700.

In one aspect, the method includes selecting the electrolyte mixture, wherein the electrolyte mixture comprises a single electrolyte solution, an aqueous mixed-salt solution, or a single salt solution. In another aspect, the method includes selecting the electrolyte mixture, wherein the electrolyte mixture is selected from Table 1, Table 2 or Table 3. In another aspect, the activity coefficient (γᵢ) for the electrolyte mixture is applicable to an entire concentration range from an infinite dilution to a pure salt. In another aspect, the activity coefficient (γᵢ) for the electrolyte mixture is accurate over a temperature range of 273 to 373 K. In another aspect, there are no mixing rules required with any ion-specific association parameters. In another aspect, the activity coefficient (γᵢ) for the electrolyte mixture is calculated using an association electrolyte model comprising:

$$ln {γ}_{i} = ln {γ}_{i}^{ASC} + ln {γ}_{i}^{PDH} + ln {γ}_{i}^{LC}$$

where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory. In another aspect, the association interactions between any species that associate (γᵢ^{ASC}) is calculated using:

$$Ln {γ}_{i}^{ASC} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ]$$

where: superscripts a and d represent electron acceptor site and electron donor site, respectively, Nᵢ is the number of association sites, X_{i,mx} and X_{i,pr} are the unbonded site fractions in the electrolyte mixture and the pure component i, respectively, ρ_{i,mx} and ρ_{i,pr} are the dimensionless molar densities of association sites in the electrolyte mixture and the pure component i, respectively, and rᵢ is the normalized Bondi's volume parameters. In another aspect, wherein rᵢ is specified as 0.76 for water and is assumed to be constants at 0.76 for all the ions. In another aspect, wherein the unbonded site fractions in the electrolyte mixture (X_{i,mx}^{a} and X_{i,mx}^{d}) and the pure component (X_{i,pr}^{a} and X_{i,pr}^{d}) are calculated as:

$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$

$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$

$${X}_{i , pr}^{a} = \frac{1}{1 + {ρ}_{i , pr}^{d} {X}_{i , pr}^{d} {Δ}^{{a}_{i} {d}_{i}}} , and$$

$${X}_{i , pr}^{d} = \frac{1}{1 + {ρ}_{i , pr}^{a} {X}_{i , pr}^{a} {Δ}^{{a}_{i} {d}_{i}}} .$$

In another aspect, wherein the dimensionless molar densities of association sites in the electrolyte mixture (ρ_{i,mx}^{a} and ρ_{i,mx}^{d}) are calculated from the densities in the pure component (ρ_{i,pr}^{a} and ρ_{i,pr}^{d}) and a mole fraction of species i (xᵢ) as:

$${ρ}_{i , mx}^{a} = \frac{{N}_{i}^{a} {x}_{i}}{{\sum }_{j} {r}_{j} {x}_{j}} ,$$

$${ρ}_{i , mx}^{d} = \frac{{N}_{i}^{d} {x}_{i}}{{\sum }_{j} {r}_{j} {x}_{j}} ,$$

$${ρ}_{i , pr}^{a} = \frac{{N}_{i}^{a}}{{r}_{i}} , and$$

$${ρ}_{i , pr}^{d} = \frac{{N}_{i}^{d}}{{r}_{i}} .$$

In another aspect, wherein chemical process or product comprises batteries, crystallization, desalination, distillation, gas refining, ion exchange, petroleum refining, or water processing.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of” As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Loehe J R, Donohue M D. Recent advances in modeling thermodynamic properties of aqueous strong electrolyte systems. AIChE Journal. 1997; 43 (1): 180-195.
2. Pitzer K S. Thermodynamics of electrolytes. I. Theoretical basis and general equations. Journal of Physical Chemistry. 1973, 77 (2): 268-277.
3. Pitzer K S, Simonson J M. Thermodynamics of multicomponent, miscible, ionic systems: theory and equations. Journal of Physical Chemistry. 1986; 90 (13): 3005-3009.
4. Pitzer K S, Wang P, Rard J A, Clegg S L. Thermodynamics of electrolytes. 13. Ionic strength dependence of higher-order terms; equations for CaCl2 and MgCl2. Journal of Solution Chemistry. 1999; 28 (4): 265-282.
5. Chen C-C, Britt H, Boston J, Evans L. Local composition model for excess Gibbs energy of electrolyte systems. Part I: single solvent, single completely dissociated electrolyte systems. AIChE Journal. 1982; 28 (4): 588-596.
6. Chen C-C, Evans L B. A local composition model for the excess Gibbs energy of aqueous electrolyte systems. AIChE Journal. 1986; 32 (3): 444-454.
7. Chen C-C, Song Y. Generalized electrolyte-NRTL model for mixed-solvent electrolyte systems. AIChE Journal 2004; 50 (8): 1928-1941.
8. Tanveer S, Chen C-C. A comprehensive thermodynamic model for high salinity produced waters. AIChE Journal. 2019; 66 (1): e16818.
9. Hamer W J, Wu Y-C. Osmotic coefficients and mean activity coefficients of uni-univalent electrolytes in water at 25° C. Journal of Physical and Chemical Reference Data. 1972; 1 (4): 1047-1099.
10. Stokes R H, Robinson R A. Ionic hydration and activity in electrolyte solutions. Journal of the American Chemical Society. 1948; 70 (5): 1870-1878.
11. Chen C-C, Mathias P M, Orbey H. Use of hydration and dissociation chemistries with the electrolyte-NRTL model. AIChE Journal. 1999; 45 (7): 1576-1586.
12. Simonin J P, Krebs S, Kunz W. Inclusion of ionic hydration and association in the MSA-NRTL model for a description of the thermodynamic properties of aqueous ionic solutions: Application to solutions of associating acids. Industrial and Engineering Chemistry Research. 2006; 45 (12): 4345-4354.
13. Schönert H. The Debye-Hückel theory for hydrated ions. I. Osmotic and activity coefficients of binary aqueous solutions of some 1:1 Electrolytes at 25° C. Berichte der Bunsengesellschaft für physikalische Chemie. 1990; 94 (6): 658-664.
14. Lu X, Maurer G. Model for describing activity coefficients in mixed electrolyte aqueous solutions. AIChE Journal. 1993; 39 (9): 1527-1538.
15. Balomenos E, Panias D, Paspaliaris I. A semi-empirical hydration model (SEHM) for describing aqueous electrolyte solutions. I. Single strong electrolytes at 25° C. Fluid Phase Equilibria. 2006; 243 (1-2): 29-37.
16. Schonert H. The Debye-Hückel theory for hydrated ions. II. Osmotic and activity coefficients of ternary aqueous solutions of hydrogen and alkali halogenides at 25° C. Berichte der Bunsengesellschaft für physikalische Chemie. 1990; 94 (6): 664-670.
17. Balomenos E, Panias D, Paspaliaris I. A semi-empirical hydration model (SEHM) for describing aqueous electrolyte solutions. II. Mixing rules for multiple electrolyte systems at 25° C. Fluid Phase Equilibria. 2006; 247 (1-2): 1-7.
18. Wertheim M S. Fluids with highly directional attractive forces. I. Statistical thermodynamics. Journal of Statistical Physics. 1984; 35 (1-2): 19-34.
19. Chapman W G, Gubbins K E, Jackson G, Radosz M. New reference equation of state for associating liquids. Industrial and Engineering Chemistry Research. 1990; 29 (8): 1709-1721.
20. Gross J, Sadowski G. Perturbed-Chain SAFT: An equation of state based on a perturbation theory for chain molecules. Industrial and Engineering Chemistry Research. 2001; 40 (4): 1244-1260.
21. Kontogeorgis G M, Michelsen M L, Folas G K, Derawi S, VonSolms N, Stenby E H. Ten Years with the CPA (Cubic-Plus-Association) equation of state. Part 1. Pure compounds and self-associating systems. Industrial and Engineering Chemistry Research. 2006; 45 (14): 4855-4868.
22. Galindo A, Gil-Villegas A, Jackson G, Burgess A N. SAFT-VRE: Phase behavior of electrolyte solutions with the statistical associating fluid theory for potentials of variable range. Journal of Physical Chemistry B. 1999; 103 (46): 10272-10281.
23. Cameretti L F, Sadowski G, Mollerup J M. Modeling of aqueous electrolyte solutions with Perturbed-Chain Statistical Associated Fluid Theory. Industrial and Engineering Chemistry Research. 2005; 44 (9): 3355-3362.
24. Maribo-Mogensen B, Thomsen K, Kontogeorgis G M. An electrolyte CPA equation of state for mixed solvent electrolytes. AIChE Journal. 2015; 61 (9): 2933-2950.
25. Held C, Reschke T, Mohammad S, Luza A, Sadowski G. ePC-SAFT revised. Chemical Engineering Research and Design. 2014; 92 (12): 2884-2897.
26. Song Y, Chen C-C. Symmetric electrolyte nonrandom two-liquid activity coefficient model. Industrial and Engineering Chemistry Research. 2009; 48 (16): 7788-7797.
27. Fu Y H, Orbey H, Sandler S I. Prediction of vapor-liquid equilibria of associating mixtures with UNIFAC models that include association. Industrial and Engineering Chemistry Research. 1996; 35 (12): 4656-4666.
28. Michelsen M L, Hendriks E M. Physical properties from association models. Fluid Phase Equilibria. 2001; 180 (1-2): 165-174.
29. Hao Y, Chen C-C. Nonrandom two-liquid segment activity coefficient model with association theory. Industrial and Engineering Chemistry Research. 2019; 58 (28): 12773-12786.
30. Hao Y, Chen C-C. Nonrandom two-liquid activity coefficient model with association theory. AIChE Journal. 2020; 67 (1): e17061.
31. Luck W A P. A model of hydrogen-bonded liquids. Angewandte Chemie International Edition in English. 1980; 19 (1): 28-41.
32. Bockris J O M. Ionic Solvation. Quarterly Reviews, Chemical Society. 1949; 3 (2): 173-180.
33. Bockris J O M, Conway B E. Modern aspects of electrochemistry vol. 1. London: Butterworths, 1954.
34. Bockris J O M, Saluja P P S. Ionic solvation numbers from compressibilities and ionic vibration potentials measurements. Journal of Physical Chemistry. 1972; 76 (15): 2140-2151.
35. Stoyanov E S, Stoyanova I V, Reed C A. The unique nature of H⁺ in water. Chemical Science. 2011; 2 (3): 462-472.
36. Stoyanov E S, Stoyanova I V, Reed C A. The structure of the hydrogen ion (Haq+) in water. Journal of the American Chemical Society. 2010: 132 (5): 1484-1485.
37. Hay B P, Dixon D A, Bryan J C, Moyer B A. Crystallographic evidence for oxygen acceptor directionality in oxyanion hydrogen bonds. Journal of the American Chemical Society. 2002; 124 (2): 182-183.
38. Gao B, Liu Z F. A first principles study on the solvation and structure of SO42-(H2O) n, n=6-12. Journal of Chemical Physics. 2004; 121 (17): 8299-8306.
39. Hay B P, Gutowski M, Dixon D A, Garza J, Vargas R, Moyer B A. Structural criteria for the rational design of selective ligands: Convergent hydrogen bonding sites for the nitrate anion. Journal of the American Chemical Society. 2004: 126 (25): 7925-7934.
40. Buchner R, Capewell S G, Hefter G, May P M. Ion-pair and solvent relaxation processes in aqueous Na₂SO₄ solutions. Journal of Physical Chemistry B. 1999; 103 (7): 1185-1192.
41. Buchner R, Chen T, Hefter G. Complexity in “simple” electrolyte solutions: Ion pairing in MgSO₄ (aq). Journal of Physical Chemistry B. 2004; 108 (7): 2365-2375.
42. Wachter W, Fernandez Š, Buchner R, Hefter G. Ion association and hydration in aqueous solutions of LiCl and Li₂SO₄ by dielectric spectroscopy. Journal of Physical Chemistry B. 2007; 111 (30): 9010-9017.
43. Friesen S, Hefter G, Buchner R. Cation hydration and ion pairing in aqueous solutions of MgCl₂ and CaCl₂. Journal of Physical Chemistry B. 2019; 123 (4): 891-900.
44. Marcus Y, Hefter G. Ion pairing. Chemical Reviews. 2006; 106 (11): 4585-4621.
45. Van DerVegt NFA, Haldrup K, Roke S, Zheng J, Lund M, Bakker H J. Water-mediated ion pairing: occurrence and relevance. Chemical Reviews. 2016; 116 (13): 7626-7641.
46. Atkinson G, Petrucci S. Ion association of magnesium sulfate in water at 25°. Journal of Physical Chemistry. 1966, 70 (10): 3122-3128.
47. Collins K D. Charge density-dependent strength of hydration and biological structure. Biophysical Journal. 1997; 72:65-76.
48. Collins K D, Neilson G W, Enderby J E. Ions in water: Characterizing the forces that control chemical processes and biological structure. Biophysical Chemistry. 2007; 128 (2-3): 95-104.
49. Harned H S, Owen B O. The physical chemistry of electrolytic solutions (3rd edition). New York: Reinhold Pub. Corp., 1958.
50. May P M, Rowland D. Thermodynamic modeling of aqueous electrolyte systems: Current status. Journal of Chemical and Engineering Data 2017; 62 (9): 2481-2495.
51. Vega R, Vera J H. Phase equilibria of concentrated aqueous solutions containing volatile strong electrolytes. The Canadian Journal of Chemical Engineering. 1976; 54 (3): 245-248.
52. Marcus Y. Ionic dissociation of aqueous hydrobromic acid. Part 1. Estimate from vapour pressure and activity coefficient data. Journal of the Chemical Society, Faraday Transactions 1: Physical Chemistry in Condensed Phases. 1979; 75:1715-1727.
53. Goldberg R N. Evaluated activity and osmotic coefficients for aqueous solutions: thirty-six uni-bivalent electrolytes. Journal of Physical and Chemical Reference Data. 1981; 10 (3): 671-764.
54. Tang I N, Munkelwitz H R, Wang N. Water activity measurements with single suspended droplets: The NaCl—H₂O and KCl—H₂O systems. Journal of Colloid And Interface Science. 1986; 114 (2): 409-415.
55. Moore Y C, Hamer W J. Revised values of the osmotic coefficients and mean activity coefficients of sodium nitrate in water at 25° C. Journal of Physical and Chemical Reference Data. 1980; 9 (2): 513-518.
56. Goldberg R N, Nuttall R L. Evaluated activity and osmotic coefficients for aqueous solutions: The alkaline earth metal halides. Journal of Physical and Chemical Reference Data. 1978; 7 (1): 263-310.
57. Robinson R A, Stokes R H. Electrolyte solutions (2nd edition). New York: Dover Publications, Inc., 2002.
58. Rard J A, Miller D G. Isopiestic determination of the osmotic coefficients of aqueous Na₂SO₄, MgSO₄, and Na₂SO₄—MgSO₄ at 25° C. Journal of Chemical and Engineering Data. 1981; 26 (1): 33-38.
59. Stokes R H. A thermodynamic study of bivalent metal halides in aqueous solution. Part XVII-revision of data for all 2:1 and 1:2 electrolytes at 25°, and discussion of results. Transactions of the Faraday Society. 1948:44:295-307.
60. Khoo K H, Chan C, Lim T K. Thermodynamics of electrolyte solutions. The system HCl+CaCl₂+H₂O at 298.15 K. Journal of Solution Chemistry. 1977; 6 (10): 651-662.
61. Roy R N, Gibbons J J, Ovens L K, Bliss G A, Hartley J J. Activity Coefficients for the system HCl+CaCl₂)+H2O at various temperatures. Journal of the Chemical Society, Faraday Transactions 1. 1981; 78:1405-1422.
62. Hawkins J E. The activity coefficients of hydrochloric acid in uni-univalent chloride solutions at constant total molality. Journal of the American Chemical Society. 1932; 54 (12): 4480-4487.
63. Khoo K H, Chan C Y, Lim T K. Activity coefficients in binary electrolyte mixtures HCl+MgCl₂+H₂O at 298.15 K. Journal of Solution Chemistry. 1977; 6 (12): 855-864.
64. Khoo K H, Lim T-K, Chan C—Y. Activity coefficients for the system HBr+CaBr₂+H₂O at 298.15 K. Journal of the Chemical Society, Faraday Transactions 1: Physical Chemistry in Condensed Phases. 1979; 75:1067-1072.
65. Robinson R A, Bower V E. Thermodynamics of the ternary system: Water-sodium chloride-barium chloride at 25° C. Journal of Research of the National Bureau of Standards Section A: Physics and Chemistry. 1965; 69A (1): 19-27.
66. Robinson R A, Bower V E. Properties of aqueous mixtures of pure salts. Thermodynamics of the ternary system: Water-sodium chloride-calcium chloride at 25 C. Journal of Research of the National Bureau of Standards Section A: Physics and Chemistry. 1966; 70A (4): 313.
67. Robinson R A. The osmotic properties of aqueous sodium chloride-cesium chloride mixtures at 25°. Journal of the American Chemical Society. 1952; 74 (23): 6035-6036.
68. Robinson R A. Activity coefficients of sodium chloride and potassium chloride in mixed aqueous solutions at 25°. Journal of Physical Chemistry. 1961; 65 (4): 662-667.
69. Bezboruah C P, Covington A K, Robinson R A. Excess Gibbs energies of aqueous mixtures of alkali metal chlorides and nitrates. The Journal of Chemical Thermodynamics. 1970; 2 (3): 431-437.
70. Robinson R A, Platford R F, Childs C W. Thermodynamics of aqueous mixtures of sodium chloride, potassium chloride, sodium sulfate, and potassium sulfate at 25° C. Journal of Solution Chemistry. 1972; 1 (2): 167-172.
71. Robinson R A, Wood R H, Reilly P J. Calculation of excess Gibbs energies and activity coefficients from isopiestic measurements on mixtures of lithium and sodium salts. The Journal of Chemical Thermodynamics. 1971; 3 (4): 461-471.
72. Robinson R A, Lim C K. The osmotic properties of some aqueous salt mixtures at 25°. Transactions of the Faraday Society. 1953; 49:1144-1147.
73. Wu Y C, Rush R M, Scatchard G. Osmotic and activity coefficients for binary mixtures of sodium chloride, sodium sulfate, magnesium sulfate, and magnesium chloride in water at 25°. I. Isopiestic measurements on the four systems with common ions. The Journal of Physical Chemistry. 1968; 72 (12): 4048-4053.
74. Rard J A, Miller D G. Isopiestic determination of the osmotic and activity coefficients of aqueous mixtures of NaCl and MgCl₂ at 25° C. Journal of Chemical and Engineering Data. 1987; 32 (1): 85-92.
75. Haghtalab A, Vera J H. Mean activity coefficients in the ternary NaCl—NaNO3-H₂O and NaBr—NaNO₃—H₂O systems at 298.15 K. Journal of Chemical and Engineering Data. 1991; 36 (3): 332-340.
76. Robinson R A, Bower V E. Properties of aqueous mixtures of pure salts: thermodynamics of the ternary system water-potassium chloride-barium chloride at 25° C. Journal of Research of the National Bureau of Standards Section A: Physics and Chemistry. 1965; 69A (5): 439-448
77. Robinson R A. The osmotic properties of aqueous caesium chloride+potassium chloride and caesium chloride+lithium chloride mixtures at 25° C. Journal of the American Chemical Society. 1953; 49:1147-1149.
78. Downes C J. Osmotic and activity coefficients for mixtures of potassium chloride and strontium chloride in water at 298.15 K. The Journal of Chemical Thermodynamics. 1974; 6 (4): 317-323.
79. Lindenbaum S, Rush R M, Robinson R A. Osmotic and activity coefficients for mixtures of lithium chloride with barium chloride and cesium chloride with barium chloride in water at 298.15 K. The Journal of Chemical Thermodynamics. 1972; 4 (3): 381-389.
80. Robinson R A, Bower V E. Properties of aqueous mixtures of pure salts. Thermodynamics of the ternary system: Water-calcium chloride-magnesium chloride at 25° C. Journal of Research of the National Bureau of Standards Section A: Physics and Chemistry. 1966; 70A (4): 305-311.
81. Platford R F. Thermodynamics of mixed salt solutions: Excess Gibbs energies of mixing for the six ternary systems formed from aqueous MgCl₂, Mg(NO₃)₂, CaCl₂, and Ca(NO₃)₂ at 25° C. The Journal of Chemical Thermodynamics. 1971; 3 (3): 319-324.
82. Åkerlöf G, Teare J W. Thermodynamics of concentrated aqueous solutions of hydrochloric acid. Journal of the American Chemical Society. 1937; 59 (10): 1855-1868.
83. Holmes H F, Busey R H, Simonson J M, Mesmer R E, Archer D G, Wood R H. The enthalpy of dilution of HCl (aq) to 648 K and 40 MPa thermodynamic properties. The Journal of Chemical Thermodynamics. 1987; 19 (8): 863-890.
84. Gibbard H F, Scatchard G. Liquid-vapor equilibrium of aqueous lithium chloride, from 25° to 100° C. and from 1.0 to 18.5 molal, and related properties. Journal of Chemical and Engineering Data. 1973; 18 (3): 293-298. Accessed Aug. 20, 2020. https://pubs.acs.org/sharingguidelines
85. Silvester L F, Pitzer K S. Thermodynamics of geothermal brines I. Thermodynamic properties of vapor-saturated NaCl (aq) solutions from 0-300° C. Berkeley: University of California, 1976.
86. Pitzer K S, Peiper J C, Busey R H. Thermodynamic properties of aqueous sodium chloride solutions thermodynamic properties of aqueous sodium chloride solutions. Journal of Physical and Chemical Reference Data. 1984; 13 (1): 1-102.
87. Snipes H P, Manly C, Ensor D D. Heats of dilution of aqueous electrolytes: temperature dependence. Journal of Chemical and Engineering Data. 1975; 20 (3): 287-291.
88. Holmes H F, Mesmer R E. Aqueous solutions of the alkaline-earth metal chlorides at elevated temperatures. Isopiestic molalities and thermodynamic properties. Journal of Chemical Thermodynamics. 1996; 28 (12): 1325-1358.
89. Ananthaswamy J, Atkinson G. Thermodynamics of concentrated electrolyte mixtures. 5. A review of the thermodynamic properties of aqueous calcium chloride in the temperature range 273.15-373.15 K. Journal of Chemical and Engineering Data. 1985; 30 (1): 120-128.
90. Gruszkiewicz M S, Simonson J M. Vapor pressures and isopiestic molalities of concentrated CaCl₂(aq), CaBr₂ (aq), and NaCl (aq) to T=523 K. Journal of Chemical Thermodynamics. 2005; 37 (9): 906-930.

What is claimed is:

1. A computerized method for determining an activity coefficient (γᵢ) for an electrolyte mixture comprising:
providing one or more processors, a memory communicably coupled to the one or more processors and an output device communicably coupled to the one or more processors;
calculating, using the one or more processors, the activity coefficient (γᵢ) for the electrolyte mixture based on association interactions between any species that associate, long-range interactions between ions, and short-range interactions between any species;
providing the activity coefficient (γᵢ) for the electrolyte mixture to the output device; and
developing a chemical process or a product using the activity coefficient (γᵢ) for the electrolyte mixture.

2. The method of claim 1, further comprising selecting the electrolyte mixture, wherein the electrolyte mixture comprises a single electrolyte solution, an aqueous mixed-salt solution, or a single salt solution.

3. The method of claim 1, further comprising selecting the electrolyte mixture, wherein the electrolyte mixture is selected from Table 1, Table 2 or Table 3.

4. The method of claim 1, wherein the activity coefficient (γᵢ) for the electrolyte mixture is applicable to an entire concentration range from an infinite dilution to a pure salt.

5. The method of claim 1, wherein the activity coefficient (γᵢ) for the electrolyte mixture is accurate over a temperature range of 273 to 373 K.

6. The method of claim 1, wherein there are no mixing rules required with any ion-specific association parameters.

7. The method of claim 1, wherein the activity coefficient (γᵢ) for the electrolyte mixture is calculated using an association electrolyte model comprising:
$$ln {γ}_{i} = ln {γ}_{i}^{ASC} + ln {γ}_{i}^{PDH} + ln {γ}_{i}^{LC}$$
where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory.

8. The method of claim 7, wherein the association interactions between any species that associate (γᵢ^{ASC}) is calculated using:
$$Ln {γ}_{i}^{ASC} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ]$$
where: superscripts a and d represent electron acceptor site and electron donor site, respectively,
Nᵢ is the number of association sites,
X_{i,mx} and X_{i,pr} are the unbonded site fractions in the electrolyte mixture and the pure component i, respectively,
ρ_{i,mx} and ρ_{i,pr} are the dimensionless molar densities of association sites in the electrolyte mixture and the pure component i, respectively, and
rᵢ is the normalized Bondi's volume parameters.

9. The method of claim 8, wherein n is specified as 0.76 for water and is assumed to be constants at 0.76 for all the ions.

10. The method of claim 8, wherein the unbonded site fractions in the electrolyte mixture (X_{i,mx}^{a} and X_{i,mx}^{d}) and the pure component (X_{i,pr}^{a} and X_{i,pr}^{d}) are calculated as:
$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$
$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$
$${X}_{i , pr}^{a} = \frac{1}{1 + {ρ}_{i , pr}^{d} {X}_{i , pr}^{d} {Δ}^{{a}_{i} {d}_{i}}} , and$$
$${X}_{i , pr}^{d} = \frac{1}{1 + {ρ}_{i , pr}^{a} {X}_{i , pr}^{a} {Δ}^{{a}_{i} {d}_{i}}} .$$

11. The method of claim 8, wherein the dimensionless molar densities of association sites in the electrolyte mixture (ρ_{i,mx} and ρ_{i,mx}^{d}) are calculated from the densities in the pure component (ρ_{i,pr} and ρ_{i,pr}^{d}) and a mole fraction of species i (xᵢ) as:
$${ρ}_{i , mx}^{a} = \frac{{N}_{i}^{a} {x}_{i}}{{\sum }_{j} {r}_{j} {x}_{j}} ,$$
$${ρ}_{i , mx}^{d} = \frac{{N}_{i}^{d} {x}_{i}}{{\sum }_{j} {r}_{j} {x}_{j}} ,$$
$${ρ}_{i , pr}^{a} = \frac{{N}_{i}^{a}}{{r}_{i}} , and$$
$${ρ}_{i , pr}^{d} = \frac{{N}_{i}^{d}}{{r}_{i}} .$$

12. The method of claim 1, wherein the chemical process or product comprises batteries, crystallization, desalination, distillation, gas refining, ion exchange, petroleum refining, or water processing.

13. The chemical process or the product developed in accordance with claim 1.

14. A system for determining an activity coefficient (γᵢ) for an electrolyte mixture comprising:
at least one input/output interface;
a data storage;
one or more processors communicably coupled to the at least one input/output interface and the data storage, wherein the one or more processors calculate the activity coefficient (γᵢ) for the electrolyte mixture based on association interactions between any species that associate, long-range interactions between ions, and short-range interactions between any species, and provide the activity coefficient (γᵢ) for the electrolyte mixture to the output device; and
wherein a chemical process or a product is developed using the activity coefficient (γᵢ) for the electrolyte mixture.

15. The system of claim 14, wherein the electrolyte mixture comprises a single electrolyte solution, an aqueous mixed-salt solution, or a single salt solution.

16. The system of claim 14, wherein the electrolyte mixture is selected from Table 1, Table 2 or Table 3.

17. The system of claim 14, wherein the activity coefficient (γᵢ) for the electrolyte mixture is applicable to an entire concentration range from an infinite dilution to a pure salt.

18. The system of claim 14, wherein the activity coefficient (γᵢ) for the electrolyte mixture is accurate over a temperature range of 273 to 373 K.

19. The system of claim 14, wherein there are no mixing rules required with any ion-specific association parameters.

20. The system of claim 14, wherein the activity coefficient (γᵢ) for the electrolyte mixture is calculated using an association electrolyte model comprising:
$$ln {γ}_{i} = ln {γ}_{i}^{ASC} + ln {γ}_{i}^{PDH} + ln {γ}_{i}^{LC}$$
where γᵢ^{ASC} is the association interactions between any species that associate, γᵢ^{PDH} is the long-range interactions between ions calculated with a Pitzer-Debye-Hückel equation, γᵢ^{LC} is the short-range interactions between any species derived from a local composition theory.

21. The system of claim 20, wherein the association interactions between any species that associate (γᵢ^{ASC}) is calculated using:
$$Ln {γ}_{i}^{ASC} = {N}_{i}^{a} [ ln ( \frac{{X}_{i , mx}^{a}}{{X}_{i , pr}^{a}} ) + \frac{{X}_{i , pr}^{a} - 1}{2} ] + {N}_{i}^{d} [ ln ( \frac{{X}_{i , mx}^{d}}{{X}_{i , pr}^{d}} ) + \frac{{X}_{i , pr}^{d} - 1}{2} ] + {r}_{i} {\sum }_{j} [ {ρ}_{j , mx}^{a} ( \frac{1 - {X}_{j , mx}^{a}}{2} ) + {ρ}_{j , mx}^{d} ( \frac{1 - {X}_{j , mx}^{d}}{2} ) ]$$
where: superscripts a and d represent electron acceptor site and electron donor site, respectively,
Nᵢ is the number of association sites,
X_{i,mx} and X_{i,pr} are the unbonded site fractions in the electrolyte mixture and the pure component i, respectively,
ρ_{i,mx} and ρ_{i,pr} are the dimensionless molar densities of association sites in the electrolyte mixture and the pure component i, respectively, and
rᵢ is the normalized Bondi's volume parameters.

22. The system of claim 21, wherein rᵢ is specified as 0.76 for water and is assumed to be constants at 0.76 for all the ions.

23. The system of claim 21, wherein the unbonded site fractions in the electrolyte mixture (X_{i,mx}^{a} and X_{i,mx}^{d}) and the pure component (X_{i,pr}^{a} and X_{i,pr}^{d}) are calculated as:
$${X}_{i , mx}^{a} = \frac{1}{1 + {\sum }_{j} {ρ}_{j , mx}^{d} {X}_{j , mx}^{d} {Δ}^{{a}_{i} {d}_{j}}} ,$$
$${X}_{i , mx}^{d} = \frac{1}{1 + {\sum }_{j} {ρ}_{j , mx}^{a} {X}_{j , mx}^{a} {Δ}^{{a}_{j} {d}_{i}}} ,$$
$${X}_{i , pr}^{a} = \frac{1}{1 + {ρ}_{i , pr}^{d} {X}_{i , pr}^{d} {Δ}^{{a}_{i} {d}_{i}}} , and$$
$${X}_{i , pr}^{d} = \frac{1}{1 + {ρ}_{i , pr}^{a} {X}_{i , pr}^{a} {Δ}^{{a}_{i} {d}_{i}}} .$$

24. The system of claim 21, wherein the dimensionless molar densities of association sites in the electrolyte mixture (ρ_{i,mx}^{a} and ρ_{i,mx}^{d}) are calculated from the densities in the pure component (ρ_{i,pr}^{a} and ρ_{i,pr}^{d}) and a mole fraction of species i (xᵢ) as:
$${ρ}_{i , mx}^{a} = \frac{{N}_{i}^{a} {x}_{i}}{{\sum }_{j} {r}_{j} {x}_{j}} ,$$
$${ρ}_{i , mx}^{d} = \frac{{N}_{i}^{d} {x}_{i}}{{\sum }_{j} {r}_{j} {x}_{j}} ,$$
$${ρ}_{i , pr}^{a} = \frac{{N}_{i}^{a}}{{r}_{i}} , and$$
$${ρ}_{i , pr}^{d} = \frac{{N}_{i}^{d}}{{r}_{i}} .$$

25. The system of claim 14, wherein the chemical process or product comprises batteries, crystallization, desalination, distillation, gas refining, ion exchange, petroleum refining, or water processing.

26. A computer program embodied on a non-transitory computer readable storage medium that is executed using one or more processors for determining an activity coefficient (γᵢ) for an electrolyte mixture comprising:
a code segment that calculates the activity coefficient (γᵢ) for the electrolyte mixture based on association interactions between any species that associate, long-range interactions between ions, and short-range interactions between any species;
a code segment that provides the activity coefficient (γᵢ) for the electrolyte mixture to the output device; and
wherein a chemical process or a product is developed using the activity coefficient (γᵢ) for the electrolyte mixture.
